(19) United States
(12) Patent Application Publication
(10) Pub. No.: US 2022/0211062 A1
(43) Pub. Date: Jul. 7, 2022

(54) METHOD FOR PRODUCING MILK PRODUCTS WITH MODIFIED FIRMNESS AND/OR GELATION TIME AND PRODUCTS OBTAINED

(71) Applicant: Chr. Hansen A/S, Hoersholm (DK)

(72) Inventors: Surender Kumar DHAYAL, Hoersholm (DK); Martin LUND, Hoersholm (DK); Henrik BISGAARD-FRANTZEN, Hoersholm (DK); Johannes Maarten VAN DEN BRINK, Hoersholm (DK)

(73) Assignee: Chr. Hansen A/S, Hoersholm (DK)

(21) Appl. No.: 17/611,416

(22) PCT Filed: May 15, 2020
(86) PCT No.: PCT/EP2020/063651

§ 371 Date: Nov. 15, 2021

(30) Foreign Application Priority Data
May 16, 2019 (EP) 19174937.3

(51) Int. Cl. A23C 11/10; C12N 9/04; C12N 9/08; A23C 9/12

(52) U.S. Cl. CPC A23C 11/106; C12N 9/0006; C12N 9/0065; C12Y 111/01007; A23C 9/1216; C12Y 101/03004; C12Y 101/99018; A23C 9/1213

(57) ABSTRACT

The present invention relates to methods for producing dairy food products modified with respect to their firmness and/or gelation time comprising cross-linked protein compounds and to methods for modifying said properties of said dairy food products, as well as to said modified dairy food products obtainable or obtained by such methods. Modification is achieved by in situ generation of H2O2 via carbohydrate oxidase, preferably cellobiose oxidase (EC 1.1.99.18), and cross-linking protein-tyrosine residues in a reaction using H202 via peroxidase (EC 1.11.1.7). Adding small phenolic compounds such as preferably p-coumaric acid or vanillin allows better control of the cross-linking reaction.

### TECHNICAL FIELD

The present invention relates to methods for producing modified food products comprising cross-linked compounds and to methods for modifying properties of food products, as well as to modified food products obtainable or obtained by such methods

### BACKGROUND

Cross-linking of milk proteins using enzymes is a mild method to change the rheological (structuring) properties of the fermented milk products. In addition to structure, the stability e.g. prevention of syneresis in yoghurt can also be improved by cross-linking. There are many food grade enzymes that can be used for cross-linking milk proteins such as transglutaminase, horseradish peroxidase (HRP), lactoperoxidase (LPO), laccase, tyrosinase etc.

One major limitation of using a peroxidase (e.g. LPO or HRP) for cross-linking is related to the use of hydrogen peroxide (H₂O₂). External addition of H₂O₂ in (semi)structured dairy products such as cheese curd or setting yoghurt makes it very difficult to uniformly distribute it and leads to formation of pockets with a high concentration of H₂O₂ leading to inactivation of enzyme/culture. Moreover, the cross-links are not uniformly distributed in the product.

Therefore, alternative methods which do not rely on the external addition of H₂O₂ are needed.

### SUMMARY

The invention is as defined in the claims.

The present invention solves the problems related to the use of a peroxidase and H₂O₂ in food applications by generating H₂O₂ in-situ by using an oxidase, in particular a cellobiose oxidase (LOX), oxygen and a carbohydrate substrate such as lactose. Lactose is naturally present in milk (whey) but can be easily added to other non-milk based food products. The LOX oxidizes lactose to lactobionic acid and H₂O₂ is formed in the process (see FIG. 1). The present methods utilize this in-situ generated H₂O₂ in combination with a peroxidase enzyme (e.g. LPO or HRP) to cross-link/polymerize/modify milk proteins (caseins as well as whey proteins). The cross-links formed by peroxidase are due to covalent conjugation of phenolic residues such as tyrosines in the case of proteins leading to formation of di-tyrosine, tri-tyrosine, tetra-tyrosine and even oligo-tyrosine cross-links.

This is schematically illustrated in FIG. 1 where protein is cross-linked by the formation of oligo-tyrosine cross-links, resulting in the formation of modified polymers. Caseins for example are expected to be very good substrates for this type of cross-linking due to their disordered configuration and good accessibility of the substrate amino acids. Other proteins such as whey proteins and apo form of α-lactalbumin can also be cross-linked, polymerized and modified using the present methods; in some cases, pre-treatment such as a heat treatment, reduction of disulfide bridges or removal of multivalent ions may be required. Therefore, this method can be used for inducing new functionality in yoghurt, cheese as well as for modifying whey proteins or enzymatic processing of whey to produce value added whey fractions. The present methods are thus very useful, in particular in the context of dairy industry.

Herein is provided a method for producing a modified food product comprising at least one cross-linked compound, said method comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;

thereby obtaining a modified food product comprising at least one cross-linked compound.

Herein is also provided a method for modifying a property such as firmness and/or gelation time of a food product, comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;

thereby obtaining a modified food product having increased firmness and/or reduced gelation time compared to the firmness and/or gelation time of the substrate.

Herein is also provided a modified food product obtainable by the methods described herein.

### DESCRIPTION OF THE DRAWINGS

FIG. 1: Schematic diagram of cross-linking/polymerization of a (milk) protein that contains phenolic residues such as tyrosine, using a combination of lactose oxidase (LOX), lactose, peroxidase e.g. horseradish peroxidase (HRP) or lactoperoxidase (LPO). Covalent cross-linking yields di-tyrosine, iso-dityrosine, tri-tyrosine, iso-tri-tyrosine and pulcherosine (not shown).

FIG. 2: Gelation of model skimmed milk due to cross-linking induced by using a combination of lactose oxidase (LOX) and horseradish peroxidase (HRP). Lactose was naturally present in the milk. The blank (b1 and b2) samples do not contain any enzyme, control 1 (c1a and c1b) contain only LOX, control 2 (c2a and c2b) contain only HRP and the test samples (Ta and Tb) contains both LOX and HRP. The data shown in FIGS. 2A, 2C, 2E and 2G are for the milk without any added calcium ions, while the data shown in FIGS. 2B, 2D, 2F and 2H are for milk with added calcium ions.

FIG. 3: Gelation of real milk due to cross-linking induced by using a combination of lactose oxidase (LOX) and horseradish peroxidase (HRP). Lactose was naturally present in the milk. The blank (b1 and b2) samples do not contain any enzyme, control 1 (c1a and c1b) contain only LOX, control 2 (c2a and c2b) contain only HRP and the test samples (Ta and Tb) contains both LOX and HRP. The data shown in FIGS. 3A, 3C, 3E and 3G are for the non-homogenized milk, while the data shown in FIGS. 3B, 3D, 3F and 3H are for the homogenized milk.

FIG. 4: Gelation time of milk incubated with various combinations of added calcium ion concentration, added lactose oxidase (LOX) and horseradish peroxidase (HRP) concentration.

FIG. 5: Cross-linking/polymerization of whey proteins using a combination of lactose oxidase (LOX), lactose and horseradish peroxidase (HRP). No calcium was added to the whey (A), while the image in (B) is for whey with added calcium ions. The covalently cross-linked/polymerized whey proteins obtained from whey that was heat treated before incubation with both enzymes can be seen with M_{w}>250 kDa.

FIG. 6: Gelation time of milk incubated with various combinations of added phenolic mediator concentration, added horseradish peroxidase (HRP) concentration and fixed dosage of lactose oxidase (LOX). Y axis shows gelation time in minutes.

FIG. 7: Firmness of model yoghurt measured using texture analyzer for the control as well as yoghurt samples that were made using milk that was incubated with lactose oxidase (LOX) and horseradish peroxidase (HRP).

FIG. 8: Acidification of heat treated (72.5° C., 40 min.) milk (A). Gel firmness measured by texture analyzer for the yoghurt samples obtained at the end of acidification (B).

### DETAILED DESCRIPTION

The present invention relates to methods for producing modified food products comprising cross-linked compounds. The method comprises the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   thereby obtaining a modified food product comprising at least one cross-linked compound.

The present methods are thus useful for modifying a substrate which is a food product comprising oxygen and a carbohydrate substrate, for example lactose, and compounds that can be cross-linked in the presence of an enzyme capable of generating H₂O₂ in the substrate. The generated H₂O₂ can be used as co-substrate by a peroxidase, which catalyzes cross-linking of the first compound, thereby obtaining a modified food product. The modified food product thus comprises cross-linked compounds which may confer desirable physico-chemical properties to the food product.

Substrate

The substrate is the food product to be modified. The food product to be modified comprises a carbohydrate substrate such as lactose, and may thus be a dairy product, such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material.

The present methods can thus be used to obtain modified food products such as modified dairy products, in particular modified yogurt, quark, cheese such as soft cheese, drinking yogurt, cheese spread, skyr or milk, such as sheep milk, goat milk, buffalo milk, yak milk, lama milk, camel milk or cow milk, or a combination thereof, optionally supplemented with plant material. Dairy products will typically contain lactose and caseins, the latter being be a suitable first compound as detailed further below. The ratio of caseins and lactose may vary depending on the nature of the dairy product.

A carbohydrate substrate is required in the present methods, as it is converted to an acid which is then required for generation of H₂O₂ by the action of the lactose peroxidase. In some embodiments, the substrate comprises in the range of 0.01% to 30% w/w of carbohydrate substrate, such as 0.05%, 1%, 5%, 10%, 15%, 20%, 25% w/w, for example between 2.5 and 6% w/w, such as 4.5% w/w carbohydrate substrate.

Oxygen is also required for the action of the oxidase. The substrate comprising a carbohydrate substrate therefore also comprises oxygen. The oxygen may be naturally present in the substrate, or it may be added as is known in the art.

Carbohydrate Substrate

The carbohydrate substrate may be any carbohydrate which can be converted into a corresponding organic acid (and H₂O₂) by the action of the oxidase, which is a cellobiose oxidase or a hexose oxidase such as a glucose oxidase as described herein in detail. The carbohydrate substrate may thus be lactose, which can be converted to lactobionic acid and H₂O₂ by the action of the oxidase. In another embodiment, the carbohydrate substrate is glucose, which can be converted to gluconic acid and H₂O₂ by the action of the oxidase. In another embodiment, the carbohydrate substrate is galactose, which can be converted to galactonic acid and H₂O₂ by the action of the oxidase. In another embodiment, the carbohydrate substrate is maltose, which can be converted to maltobionic acid and H₂O₂ by the action of the oxidase. In another embodiment, the carbohydrate substrate is xylose, which can be converted to xylonic acid and H₂O₂ by the action of the oxidase. In another embodiment, the carbohydrate substrate is cellobiose, which can be converted to cellobionic acid and H₂O₂ by the action of the oxidase. In another embodiment, the carbohydrate substrate is mannose, which can be converted to mannonic acid and H₂O₂ by the action of the oxidase. In another embodiment, the carbohydrate substrate is fructose, which can be converted to fructonic acid and H₂O₂ by the action of the oxidase. Oxygen is required for the reaction, as detailed herein.

It is to be understood throughout the present disclosure that the carbohydrate substrate on which the oxidase acts may be inherently present in the product to be modified, i.e. the substrate, or it may be obtained by treating the substrate as is known in the art. For example, if the substrate is a dairy product, the substrate may be treated with lactase, whereby the lactose present in the substrate is converted to galactose and glucose, which are converted by the oxidase to galactonic acid and gluconic acid, respectively, while generating H₂O₂ in the substrate. Such additional enzymatic treatment may occur prior to step i) or concomitantly with any of steps i), ii) and iii). Preferably, such treatment is performed prior to or concomitantly with step i). Likewise, oxygen may be inherently present in the product to be modified, or it may be added thereto by treating the substrate as is known in the art.

First Compound

The substrate comprises a carbohydrate substrate such as lactose and at least one first compound. The first compound is a compound which can be cross-linked. In some embodiments, the first compound is a phenolic compound, a non-phenolic aromatic compound (i.e. a non-phenolic compound which is an aromatic compound), a compound comprising a sulfhydryl group and a compound comprising an amino group, for example a protein comprising at least one aromatic amino acid such as tyrosine.

In some embodiments, the first compound is a phenolic compound. The phenolic compound phenolic may be a plant phenolic compound, such as a phenolic compound from a grain such as a cereal, a bean such as a coffee bean, a leaf such as a tea leaf, a vegetable pulp or a vegetable peel such as from a tuberculous vegetable, or an animal phenolic compound, such as a phenolic compound from an insect, a mammal or a fish, such as a phenolic compound derived from side streams from food or feed or paper or wood processing industry. In particular, the phenolic compound may be lignin, lignosulfonate, caffeic acid, cholorogenic acid, a flavonoid, a flavonol, quercetin, rutin, tannic acid, vanillin, p-coumaric acid, ferulic acid or ABTS. In some embodiments, the phenolic compound is not lignin or lignosulfonate.

The first compound may also be a protein such as a milk protein, for example a casein or whey protein, or the protein may be a plant protein, a fish protein or an animal protein. Preferably, the primary structure of the protein contains aromatic amino acids such as tyrosine residues which are accessible for cross-linking. Proteins with disordered or random coil solution conformation (e.g. caseins) are good substrates for cross-linking. Other proteins, for example globular proteins (e.g. whey proteins), may be less amenable to cross-linking and can be pre-processed e.g. by removal of multivalent ions using chelating agents and/or by heat treatment to make them more amenable to cross-linking.

In order to achieve cross-linking of the first compound, it may be necessary to include in the method a step of pre-treatment of the substrate prior to the step of incubating the substrate with the cellobiose oxidase and the peroxidase (i.e. prior to step iii)). It may be judicious to perform the step of pre-treatment prior to contacting the substrate with the cellobiose oxidase and the peroxidase; however, the pre-treatment step may also be performed concomitantly with step ii) or iiii).

In some embodiments, in particular where the first compound is a protein, more particularly whey protein, the method thus comprises a step of pre-treatment, for example heat treatment, reduction of disulphide bridges and/or removal of multivalent ions, thereby increasing accessibility of the aromatic amino acids, as is known in the art.

The substrate may comprise a plurality of first compounds, which can be cross-linked to one another, thereby forming heteropolymers.

In some embodiments, the substrate comprises in the range of 0.01% to 30% w/w of the first compound, such as 0.05%, 1%, 5%, 10%, 15%, 20%, 25% w/w, for example between 2.5 and 6% w/w, such as 3.5% w/w of the first compound.

Accordingly, in some embodiments, the substrate comprises in the range of 0.01% to 30% w/w of a protein, such as 0.05%, 1%, 5%, 10%, 15%, 20%, 25% w/w, for example between 2.5 and 6% w/w, such as 3.5% w/w of a protein such as a milk protein, for example a casein or whey protein, of a plant protein, a fish protein or an animal protein.

Oxidase

The present methods thus rely on in situ formation of H₂O₂ by the action of an oxidase selected from a cellobiose oxidase and a hexose oxidase such as a glucose oxidase, which converts the carbohydrate substrate and oxygen to a corresponding organic acid and H₂O₂. The peroxidase can then catalyze cross-linking of the first compound using said H₂O₂ as a co-substrate to obtain a cross-linked compound.

In some embodiments, the oxidase is a cellobiose oxidase. Cellobiose oxidase is an unspecific enzyme of EC number EC 1.1.99.18, capable of catalyzing conversion of different carbohydrate substrates and oxygen into the corresponding organic acids and H₂O₂. The enzyme is unspecific, and can convert for example (in the presence of oxygen):
Lactose to lactobionic acid,
Glucose to gluconic acid,
Galactose to galactonic acid,
Maltose to maltobionic acid,
Xylose to xylonic acid,
Cellobiose to cellobionic acid,
Mannose to mannonic acid,
Fructose to fructonic acid,

While generating H₂O₂.

Cellobiose oxidase (EC 1.1.99.18) may alternatively be termed lactose oxidase (LOX) or carbohydrate oxidase, and the terms will be used interchangeably herein.

In some embodiments, the cellobiose oxidase is LactoYield® (Chr. Hansen A/S). In some embodiments, the cellobiose oxidase (EC 1.1.99.18) enzyme is an enzyme:

(i): comprising the polypeptide sequence of position 23-495 of SEQ ID NO: 2 of EP1041890B1, which starts with Gly in position 23 and ends with Lys in position 495; or

(ii): a variant of (i), wherein the variant comprises less than 20 amino acid alterations, preferably less than 10 amino acid alterations, more preferably less than 5 amino acid alterations, wherein the amino acid alterations may be preferably a substitution, a deletion or an insertion—most preferably a substitution, as compared to polypeptide sequence of (i).

Useful cellobiose oxidases are described in application “Use of cellobiose oxidase for reduction of reduction of Maillard reaction” filed by same applicant on May 24, 2018.

The cellobiose oxidase may also or alternatively naturally be present in the substrate.

In other embodiments, the oxidase is a hexose oxidase such as a glucose oxidase (EC 1.1.3.4), which can catalyze the conversion of a hexose such as glucose, and oxygen, to the corresponding organic acid, such as gluconic acid, and H₂O₂.

In some embodiments of the method, the concentration of oxidase, i.e. the cellobiose oxidase or the hexose oxidase such as the glucose oxidase, relative to the substrate is in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.

Accordingly, in some embodiments of the method where the substrate is a dairy product, the concentration of oxidase, e.g. the cellobiose oxidase or hexose oxidase such as the glucose oxidase, relative to the dairy product is in the range of 0.0001 to 15 U/g dairy product, such as 0.01 U/g dairy product, 0.05 U/g dairy product, or 0.15 U/g dairy product, for example between 0.001 and 12.5 U/g dairy product, such as between 0.005 and 10 U/g dairy product, for example between 0.01 and 7.5 U/g dairy product, such as between 0.05 and 5 U/g dairy product, for example between 0.1 and 2.5 U/g dairy product, such as between 0.15 and 1 U/g dairy product, for example between 0.25 and 0.75 U/g substrate, such as 0.5 U/g dairy product. The dairy product may be as described above, i.e. a yogurt, quark, a cheese such as a soft cheese, a drinking yogurt, a cheese spread, skyr or milk, such as soy milk, sheep milk, goat milk, buffalo milk, yak milk, lama milk, camel milk or cow milk, or a combination thereof, optionally supplemented with plant material.

In some embodiments of the method, the oxidase is a cellobiose oxidase, such as LactoYield®, and the concentration of cellobiose oxidase, e.g. the LactoYield® cellobiose oxidase, relative to the substrate is in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.

Accordingly, in some embodiments of the method where the substrate is a dairy product, the concentration of cellobiose oxidase, e.g. the LactoYield® cellobiose oxidase, relative to the dairy product is in the range of 0.0001 to 15 U/g dairy product, such as 0.01 U/g dairy product, 0.05 U/g dairy product, or 0.15 U/g dairy product, for example between 0.001 and 12.5 U/g dairy product, such as between 0.005 and 10 U/g dairy product, for example between 0.01 and 7.5 U/g dairy product, such as between 0.05 and 5 U/g dairy product, for example between 0.1 and 2.5 U/g dairy product, such as between 0.15 and 1 U/g dairy product, for example between 0.25 and 0.75 U/g substrate, such as 0.5 U/g dairy product. The dairy product may be as described above, i.e. a yogurt, quark, a cheese such as a soft cheese, a drinking yogurt, a cheese spread, skyr or milk, such as soy milk, sheep milk, goat milk, buffalo milk, yak milk, lama milk, camel milk or cow milk, or a combination thereof, optionally supplemented with plant material.

Peroxidase

The present methods require the presence of a peroxidase. Peroxidase is an enzyme of EC number EC 1.11.1.7 which can catalyze cross-linking of the first compounds using the H₂O₂ generated by the action of the oxidase, such as the cellobiose oxidase or the hexose oxidase such as the glucose oxidase, as a co-substrate. In some embodiments, the peroxidase is endogenous to the substrate, i.e. it is naturally present in the substrate. However, the peroxidase may also be added to the reaction. In embodiments where the substrate is a milk, such as sheep milk, goat milk, buffalo milk, yak milk, lama milk, camel milk or cow milk, or a combination thereof, optionally supplemented with plant material, the peroxidase can advantageously be added at the beginning of the reaction.

Cross-linking may comprise the formation of intramolecular and/or intermolecular covalent cross-links between molecules of the phenolic compound. Cross-linking may also comprise the formation of intermolecular covalent cross-links between molecules of the phenolic compound and protein molecules. In particular, cross-linking may involve the formation of oligo-tyrosine cross-links, such as di-tyrosine cross-links and/or iso-di-tyrosine cross-links. These may be formed by covalent bonds of type C—C (e.g. in di-tyrosine cross-links). Other types of covalent bonds are C—O—C bonds, C—N bonds, S—S bonds and C—S bonds. C—O—C bonds can for example be in iso-di-tyrosine cross-links; C—N bonds can for example involve a carbon on a phenolic ring of the first compound and a nitrogen within the first compound or the second compound as described below, for example on an amino chain of a protein. C—S bonds can for example involve a carbon on a phenolic ring of the first compound and a sulphur on a sulphydryl side chain of the first compound or the second compound as described below, for example a sulphur or a sulphydryl side chain of a protein. S—S bonds can occur in the case of disulphide cross-links. Cross-linking may occur within one molecule of the first compound by formation of intramolecular covalent bonds, or between one molecule of the first compound and another molecule of the first compound or of the second compound as described below, via formation of intermolecular covalent bonds.

In some embodiments, the peroxidase is lactoperoxidase. In other embodiments, the peroxidase is horseradish peroxidase. In other embodiments, the peroxidase is lignin peroxidase. In other embodiments, the peroxidase is Coprinus peroxidase. In other embodiments, the peroxidase is myeloperoxidase.

In some embodiments, the concentration of peroxidase relative to the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. In particular embodiments, the peroxidase is lactoperoxidase, and its concentration in the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. In other embodiments, the peroxidase is horseradish peroxidase, and its concentration in the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. In other embodiments, the peroxidase is lignin peroxidase, and its concentration in the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. In other embodiments, the peroxidase is Coprinus peroxidase, and its concentration in the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. In other embodiments, the peroxidase is myeloperoxidase, and its concentration in the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate. The concentration of oxidase, in particular cellobiose oxidase, for example LactoYield®, relative to the substrate in such embodiments, may be in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.

In some embodiments, the substrate is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material, and the concentration of peroxidase relative to the dairy product is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. In particular embodiments, the peroxidase is lactoperoxidase, and its concentration in the substrate is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. In other embodiments, the peroxidase is horseradish peroxidase, and its concentration in the substrate is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. In other embodiments, the peroxidase is lignin peroxidase, and its concentration in the substrate is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. In other embodiments, the peroxidase is Coprinus peroxidase, and its concentration in the substrate is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. In other embodiments, the peroxidase is myeloperoxidase, and its concentration in the substrate is in the range of 0.001 to 500 U/g dairy product, such as 5, 15, 30, or 50 U/g dairy product, for example between 0.01 and 250 U/g dairy product, such as between 0.05 and 125 U/g dairy product, for example between 0.1 and 100 U/g dairy product, such as between 0.5 and 75 U/g dairy product, for example between 1 and 50 U/g dairy product, such as between 5 and 40 U/g dairy product, for example between 10 and 30 U/g dairy product, for example 15, 20 or 25 U/g dairy product. The concentration of oxidase, in particular cellobiose oxidase, for example LactoYield®, relative to the dairy product in such embodiments may be in the range of 0.0001 to 15 U/g dairy product, such as 0.01 U/g dairy product, 0.05 U/g dairy product, or 0.15 U/g dairy product, for example between 0.001 and 12.5 U/g dairy product, such as between 0.005 and 10 U/g dairy product, for example between 0.01 and 7.5 U/g dairy product, such as between 0.05 and 5 U/g dairy product, for example between 0.1 and 2.5 U/g dairy product, such as between 0.15 and 1 U/g dairy product, for example between 0.25 and 0.75 U/g substrate, such as 0.5 U/g dairy product.

Additional Substrate

In some embodiments, the method further comprising providing an additional substrate, for example in step i), and contacting and incubating said additional substrate with the substrate comprising the first compound in steps ii) and iii). The additional substrate comprises at least one co-mediator which consists of Ca²⁺ or a second compound such as a phenolic compound, for example a protein comprising at least one aromatic residue such as tyrosine. In such embodiments, the cross-linking in step iii) comprises the formation of intermolecular covalent cross-links between molecules of the first compound and molecules of the second compound. Cross-links between molecules of the second compound may also be formed, as well as cross-links between molecules of the first compound. The addition of an additional substrate comprising a co-mediator, in particular a phenolic compound, may advantageously be used to reduce the amount of enzyme(s) needed for the reaction.

The additional substrate may be a grain hull, a grain such as a cereal grain, fruit pulp or fruit peel, a bean such as a coffee bean, a leaf such as a tea leaf, a vegetable pulp or a vegetable peel such as pulp or peel from a tuberculous vegetable, a fruit extract, a vegetable extract, a seed extract or a yeast extract.

The phenolic compound phenolic may be a plant phenolic compound, such as a phenolic compound from a grain such as a cereal, a bean such as a coffee bean, a leaf such as a tea leaf, a vegetable pulp or a vegetable peel such as from a tuberculous vegetable, or an animal phenolic compound, such as a phenolic compound from an insect, a mammal or a fish, such as a phenolic compound derived from side streams from food or feed or paper or wood processing industry. In particular, the phenolic compound may be lignin, lignosulfonate, caffeic acid, cholorogenic acid, a flavonoid, a flavonol, quercetin, rutin, tannic acid, vanillin, p-coumaric acid, ferulic acid or ABTS. In some embodiments, the phenolic compound is not lignin or lignosulfonate.

The second compound may thus be selected from the group consisting of caffeic acid, cholorogenic acid, flavonoids, flavonols, quercetin, rutin, tannic acid, vanillin, p-coumaric acid and ferulic acid, preferably vanillin and p-coumaric.

The co-mediator may be Ca²⁺, preferably the concentration of Ca²⁺ is between 0.05 and 5000 mg/L, such as between 0.1 and 4000 mg/L, for example between 10 and 3000 mg/L, such as 100 and 2500 mg/L, for example between 150 and 2000 mg/L, such as between 300 and 1500 mg/L, for example between 500 and 1000 mg/L, such as between 600 and 900 mg/L, for example between 700 and 800 mg/L.

Reaction Conditions

Step iii) of the present methods may be performed under a variety of reaction conditions. The oxidase, in particular the cellobiose oxidase or hexose oxidase such as the glucose oxidase, and the peroxidase may be provided at the concentrations described herein above.

In some embodiments, step iii) is performed at a temperature of 4° C. to 75° C., such as between 4° C. and 72° C., for example between 4° C. and 70° C., such as between 4° C. and 65° C., for example between 4° C. and 60° C., such as between 4° C. and 55° C., for example between 4° C. and 50° C., such as between 4° C. and 45° C., for example between 4° C. and 40° C., such as between 4° C. and 37° C., for example between 4° C. and 35° C., such as between 4° C. and 30° C., for example between 4° C. and 25° C., such as between 4° C. and 20° C., for example between 4° C. and 15° C., such as between 4° C. and 10° C., or such as between 10° C. and 75° C., for example between 15° C. and 75° C., such as between 20° C. and 75° C., for example between 25° C. and 75° C., such as between 30° C. and 75° C., for example between 35° C. and 75° C., such as between 37° C. and 75° C., for example between 40° C. and 75° C., such as between 45° C. and 75° C., for example between 50° C. and 75° C., such as between 55° C. and 75° C., for example between 60° C. and 75° C., such as between 65° C. and 75° C., for example between 72° C. and 75° C., such as at 75° C., 72° C., 40° C., 37° C., 25° C. or 4° C.

In some embodiments, step iii) is performed for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours.

In some embodiments, step iii) is performed at a temperature of 4° C. to 75° C., such as between 4° C. and 72° C., for example between 4° C. and 70° C., such as between 4° C. and 65° C., for example between 4° C. and 60° C., such as between 4° C. and 55° C., for example between 4° C. and 50° C., such as between 4° C. and 45° C., for example between 4° C. and 40° C., such as between 4° C. and 37° C., for example between 4° C. and 35° C., such as between 4° C. and 30° C., for example between 4° C. and 25° C., such as between 4° C. and 20° C., for example between 4° C. and 15° C., such as between 4° C. and 10° C., or such as between 10° C. and 75° C., for example between 15° C. and 75° C., such as between 20° C. and 75° C., for example between 25° C. and 75° C., such as between 30° C. and 75° C., for example between 35° C. and 75° C., such as between 37° C. and 75° C., for example between 40° C. and 75° C., such as between 45° C. and 75° C., for example between 50° C. and 75° C., such as between 55° C. and 75° C., for example between 60° C. and 75° C., such as between 65° C. and 75° C., for example between 72° C. and 75° C., such as at 75° C., 72° C., 40° C., 37° C., 25° C. or 4° C., and fora duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours.

In specific embodiments, step iii) is performed at a temperature of 75° C. for 15 seconds, or at a temperature of 72° C. for 30 seconds, or at a temperature of 40° C. for 3 to 6 hours, such as at a temperature of 40° C. for 3 hours, for 4 hours, for 5 hours or for 6 hours.

In some embodiments, the pH of the substrate in any of steps i), ii) or iii) and/or the pH of the product in step iii) is in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 75° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 72° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 40° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 37° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 25° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed at a temperature of 4° C. for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours, and at a pH in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.

In some embodiments, step iii) is performed under conditions suitable for pasteurization, which may be particularly relevant in embodiments where the substrate is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material, since pasteurization may then be performed concomitantly with step iii). The skilled person knows how to perform pasteurization.

Inactivation of Oxidase and/or Peroxidase

The present methods may further comprise a step of heating the modified food product to inactivate the oxidase and/or the peroxidase, such as heating at 90° C. for 10 minutes or heating at 141° C. for 8 seconds or heating at 72° C. for 15 seconds or heating at 63° C. for 30 minutes or any other suitable combination of temperature and time to inactivate at least one of the enzymes. In some embodiments, the oxidase is a cellobiose oxidase and this step inactivates at least the cellobiose oxidase. In other embodiments, the oxidase is a hexose oxidase such as a glucose oxidase and this step inactivates at least the hexose oxidase, such as at least the glucose oxidase. In some embodiments, the step inactivates only the peroxidase. In other embodiments, the step inactivates only the oxidase. In some embodiments, the step inactivates both the oxidase and the peroxidase.

The step of heat inactivation may be performed concomitantly with a step of sterilization (e.g. U.H.T.) treatment. This may be particularly relevant in embodiments where the substrate is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material, as the sterilization step may be performed concomitantly with step iii).

The oxidase, i.e. the cellobiose oxidase or the hexose oxidase, such as the glucose oxidase, and/or the peroxidase may also be inactivated by modifying the pH of the product. Accordingly, in some embodiments the method further comprises the step of reducing the pH of the modified food product to below 4, whereby the oxidase and/or the peroxidase is inactivated.

In some embodiments, the oxidase is a cellobiose oxidase, the activity of which is inactivated by said step. In some embodiments, the peroxidase is inactivated. In some embodiments, both cellobiose oxidase and peroxidase are inactivated.

Additional Steps

The methods may advantageously further comprise a step of fermentation. This can be desirable for example when the substrate is milk or a dairy product. The methods may thus comprise a step of fermentation, for example to ferment milk to a dairy product, and/or a step of bacterial acidification, which may be performed concomitantly with steps ii) and/or iii).

The method may also comprise a step of pasteurization or sterilization as is known in the art. This step may be performed concomitantly with step iii). This may be particularly advantageous in embodiments where the substrate is a dairy product, such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material.

Modified Food Product

Using the present methods, a modified food product is obtained. Accordingly, also provided herein is a modified food product obtainable or obtained by the methods disclosed herein.

In particular, the disclosure provides a modified food product obtainable or obtained by a method comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate.

The cellobiose oxidase may be replaced by a hexose oxidase such as a glucose oxidase, as detailed herein. The carbohydrate substrate and acid may be as described herein above.

The modified food product may be a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material.

The substrate may be a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material.

The cellobiose oxidase may be as described herein, in particular it may be LactoYield®.

The peroxidase may be endogenous or exogenous to the substrate. The peroxidase may be a lactoperoxidase or a horseradish peroxidase, as described herein.

The modified food product may comprise at least 0.001% cross-linked compound, such as at least 0.01%, such as at least 0.1%, such as at least 0.5%, such as at least 1%, such as at least 2% cross-linked compound, such as at least 5%, such as at least 10%, such as at least 20%, such as at least 30%, such as at least 40%, such as at least 50%, such as at least 60%, such as at least 70% or more, wherein the percentage is in w/w of total protein of the food product.

In some embodiments, the modified product is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material and may comprise at least 0.001% cross-linked compound, such as at least 0.01%, such as at least 0.1%, such as at least 0.5%, such as at least 1%, such as at least 2% cross-linked compound, such as at least 5%, such as at least 10%, such as at least 20%, such as at least 30%, such as at least 40%, such as at least 50%, such as at least 60%, such as at least 70% or more, wherein the percentage is in w/w of total protein of the dairy product.

In some embodiments, the food product may comprise from 0.00001 mg to 250 mg of cross-linked compound per g of food product, such as from 0.0001 to 200 mg, such as from 0.001 to 150 mg, such as from 0.01 to 100 mg, such as from 0.1 to 75 mg, such as from 0.5 to 74 mg, such as from 1 to 50, such as from 5 to 25 mg of cross-linked compound per g of food product.

In some embodiments, the modified product is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material and may comprise from 0.00001 mg to 250 mg of cross-linked compound per g of food product, such as from 0.0001 to 200 mg, such as from 0.001 to 150 mg, such as from 0.01 to 100 mg, such as from 0.1 to 75 mg, such as from 0.5 to 74 mg, such as from 1 to 50, such as from 5 to 25 mg of cross-linked compound per g dairy product.

As understood by the skilled person in the present context, the averaged degree of polymerization (DP) relates to the extent of cross-linking, for example via intermolecular or intramolecular covalent bonds as described above. The averaged degree of polymerization (DP) of the cross-linked compound may be from 2 to 100000, such as from 3 to 100000, such as from 5 to 1000, such as from 8 to 200, such as from 9 to 150, such as 100 or 125.

The formation of cross-links in the food product to be modified may result in modification of at least one property of the food product used as a substrate. In some embodiments, the modified property is gelation time and/or firmness and/or syneresis. Accordingly, in some embodiments a modified food product having a shorter gelation time and/or increased firmness and/or reduced likelihood of syneresis compared to the food product used as substrate is obtained. In some embodiments, the food product is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material.

The method may be as otherwise described in detail herein.

Novel Functionalities

The present methods may be used for a number of applications as the cross-linked compounds may have novel functionalities. For example, the present cross-linked compounds may be used for ion binding (e.g. Ca binding—preferably Calcium Phosphate (CaP) binding), encapsulation of a bioactive agent, for example encapsulation of a phytochemical such as e.g. curcumin or β-carotene), encapsulation of a molecule (e.g. enzyme such as e.g. lactase), gelation, responsive gel swelling for triggered (e.g. pH, ionic strength, temperature) release, covalent conjugation, electrostatic complex formation, or colloid stabilization (e.g. acidic stabilization, Pickering stabilization or via self-assembled structures/aggregates), or encapsulation of a microorganism such as probiotic microorganism. In the context of encapsulation of a bioactive substance, reference is made to application “Methods for encapsulation” filed on the same date and by the same applicant as the present application.

In some embodiments, a method for encapsulation of a bioactive agent may comprise the steps of:
i) Providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) Contacting said substrate with a cellobiose oxidase (EC 1.1.99.18) and with a peroxidase (EC 1.11.1.7);
iii) Incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   Wherein step i) further comprises providing a bioactive agent to be encapsulated, thereby obtaining a modified food product comprising at least one cross-linked compound encapsulating the bioactive agent.

In some embodiments, the substrate is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material. The cellobiose oxidase may be LactoYield®. The cellobiose oxidase may be replaced with a hexose oxidase such as a glucose oxidase as described herein. The peroxidase may be lactoperoxidase, horseradish peroxidase, lignin peroxidase, Coprinus peroxidase or myeloperoxidase.

The reaction conditions, in particular for step iii), may be as described herein above.

In particular, a method is provided for encapsulation of a bioactive agent, said method comprising the steps of:
i) providing a microorganism, a heteropolymer obtained by cross-linking of a first compound which is a phenolic compound with a protein comprising at least one aromatic amino acid, and a polymer, said polymer having the ability to phase separate from said heteropolymer, preferably having the ability to coacervate or to form a complex with said heteropolymer;
ii) contacting said microorganism with said heteropolymer,
iii) inducing phase separation, such as coacervation or complex coacervation, of the heteropolymer with the polymer, to obtain a continuous phase and a dispersed phase, wherein one of continuous phase and of the dispersed phase comprises heteropolymer particles encapsulating said microorganism,

thereby obtaining a product comprising heteropolymer particles encapsulating said microorganism.

In some embodiments, the substrate is a dairy product such as a yogurt, quark, a cheese such as a soft cheese, a drinking yogurt, a cheese spread, skyr or milk, such as sheep milk, goat milk, buffalo milk, yak milk, lama milk, camel milk or cow milk, or a combination thereof, optionally supplemented with plant material. The cellobiose oxidase may be LactoYield®. The peroxidase may be lactoperoxidase, horseradish peroxidase, lignin peroxidase, Coprinus peroxidase or myeloperoxidase.

The reaction conditions, in particular for step iii), may be as described herein above.

### EXAMPLES

### Example 1: Materials

The skimmed milk powder (SMP) used was from Arlafoods. The calcium chloride (CaCl₂)) concentrate was at 50% w/v, density=1.36 g/mL. Tri-sodium citrate dihydrate was from Merck. The lactose oxidase/cellobiose oxidase (LOX) used was the formulated product sold by Chr. Hansen (LactoYield®, activity=15 LOX U/g). The horseradish peroxidase (HRP) was from Sigma Aldrich (P8125, activity=50 kU/g, where 1-unit forms 1 mg purpurogallin from pyrogallol in 20 s at pH 6.0 at 20° C.). The phenolic compounds used as oxidation mediators were: Vanillin (Sig-ma W310727, Mw 152.15 Da, in EtOH), ABTS (Roche 10102946001, Mw 548.7 Da), Ferulic acid (Sigma 128708, Mw 194.18 Da), and p-Coumaric acid (Sigma C9008, Mw 164.16 Da in EtOH).

### Example 2: Methods

Solution Preparation

Model milk was prepared by dissolving 1.1 g of skimmed milk powder (SMP) in 10 mL of MQ-water (18.2 MO cm), which also contained 10 μL of CaCl₂) (50% w/v). The solution was stirred on a magnetic stirrer for 30 min. at room temperature, followed by rest for another 15-20 min. at room temperature. In the case of SMP model milk without Ca²⁺ ions, CaCl₂) was not added to the water used for dissolving SMP powder.

For making 0.5 M stock solution of tri-sodium citrate, accurately weigh 7.35 g of tri-sodium citrate dihydrate powder and transfer it to a 50 mL volumetric flask. 40-45 mL MQ-Water was added until the salt was dissolved and the volume was made up to 50 mL. The stock solution was diluted to 10 mM concentration with MQ-water.

Stock Solution of HRP was prepared by accurately weighing 4 mg of HRP powder and adding 40 μL of MQ-Water to the powder.

Enzymatic Cross-Linking

1 mL of model milk (with or without Ca²⁺ ions) was placed in an Eppendorf tube of 2 mL capacity. See table 1 for the details of the cross-linking reaction. 10 μL of MQ-Water were added to control-1 and control-2 tubes. 20 μL of MQ-Water were added to Blank tubes. 10 μL of HRP stock solution were added to Test and control-2 tubes. The 8 Eppendorf tubes were placed in a thermomixer and incubate at 40° C. for 15 minutes. 10 μL of LOX stock solution was added to Test and control-1 tubes (time 0). After 1 h of incubation at 40° C., 50 μL of the solution were taken from the reaction tube and added to 950 μL of the trisodium citrate buffer (10 mM). The diluted sample was heated at 90° C. for 10 minutes and then cooled over ice and stored at 4° C. till further analysis. This ‘diluted sample’ was used for further analytical analysis such as SDS-PAGE, fluorescence and absorbance measurement. After collection of the 4 h time point sample, the Eppendorf tubes with the remaining solution were inverted and photographed. If there is gel formation, the sample does not flow down after the Eppendorf is inverted. The enzymes were heat inactivated (90° C., 10 min.), the milk cooled over ice to room temperature and the pH of the milks in all the tubes was measured.

Enzymatic cross-linking of 100 μl of skimmed milk was also performed in a 96 well plate or microtiter plate (MTP) to determine the gelation time in a high throughput manner at 40° C. and in duplicates. Measurement of optical density (OD) at 800 nm was used to determine the gelation time, after which there was a sharp increase in the OD. Experiments were performed using a factorial design to the effect of varying the dosage of Ca²⁺, LOX and HRP in the range given below:

Ca²⁺={0, 15, 30, 50 g/100 L}

LOX={0.01, 0.05, 0.15 U/ml}

HRP={5, 15, 30, 50 U/ml}

Data analysis was done with a R-script allowing fast evaluation of data.

TABLE 1
Details of cross-linking reaction conditions for model (SMP) milk

| | | | Control-1 | | Control-2 | | | |
|---|---|---|---|---|---|---|---|---|
| | Test | | (LOX) | | (HRP) | | Blank | |
| Replicates | 2X | | 2X | | 2X | | 2X | |
| | Label: | | Label: | | Label: | | Label: | |
| | Ta & Tb | | C1a & C1b | | C2a & C2b | | B1 & B2 | |
| SMP Milk | 1 | mL | 1 | mL | 1 | mL | 1 | mL |
| HRP stock | 10 | μL | — | | 10 | μL | — | |
| LOX stock | 10 | μL | 10 | μL | — | | — | |
| MQ-Water | — | | 10 | μL | 10 | μL | 20 | μL |
| Temperature | 40° | C. | 40° | C. | 40° | C. | 40° | C. |

| | | | | |
|---|---|---|---|---|
| Samples | 1 h, 2 h, | 1 h, 2 h, | 1 h, 2 h, | 1 h, 2 h, |
| | 3 h and 4 h | 3 h and 4 h | 3 h and 4 h | 3 h and 4 h |
| Enzyme | 90° C., | 90° C., | 90° C., | 90° C., |
| Inactivation | 10 min. | 10 min. | 10 min. | 10 min. |

TABLE 2
Details of cross-linking reaction conditions
for model whey with LOX-HRP

| | | | Control-1 | | Control-2 | | | |
|---|---|---|---|---|---|---|---|---|
| | Test | | (LOX) | | (HRP) | | Blank | |
| Replicates | 2X | | 2X | | 2X | | 2X | |
| | Label: | | Label: | | Label: | | Label: | |
| | Ta & Tb | | C1a & C1b | | C2a & C2b | | B1 & B2 | |
| Whey +/− Ca²⁺ | 1 | mL | 1 | mL | 1 | mL | 1 | mL |
| HRP stock | 10 | μL | — | | 10 | μL | — | |
| LOX stock | 10 | μL | 10 | μL | — | | — | |
| MQ-Water | — | | 10 | μL | 10 | μL | 20 | μL |
| Temp. | 40° | C. | 40° | C. | 40° | C. | 40° | C. |

TABLE 3
Details of cross-linking reaction conditions for homogenized
and non-homogenized milk (a) and the measured pH values
before and after enzymatic incubation (b).
(a)

| | | | Control-1 | | Control-2 | | | |
|---|---|---|---|---|---|---|---|---|
| | Test | | (LOX) | | (HRP) | | Blank | |
| Replicates | 2X | | 2X | | 2X | | 2X | |
| | Label: | | Label: | | Label: | | Label: | |
| | Ta & Tb | | C1a & C1b | | C2a & C2b | | B1 & B2 | |
| Milk (3.5% fat) | 1 | mL | 1 | mL | 1 | mL | 1 | mL |

| | | | | | | |
|---|---|---|---|---|---|---|
| HRP stock | 10 | μL | — | 10 | μL | — |

| | | | | | | |
|---|---|---|---|---|---|---|
| LOX stock | 10 | μL | 10 | μL | — | — |

| | | | | | | | |
|---|---|---|---|---|---|---|---|
| MQ-Water | — | 10 | μL | 10 | μL | 20 | μL |

(b)
pH measurement

| Sample | Homogenized (H) | Non-homogenized (N) |
|---|---|---|
| Milk | 6.75 | |
| Milk | | 6.78 |
| Ta | 6.01 | 6.12 |
| Tb | 5.94 | 6.2 |
| C1a | 6.17 | 6.33 |
| C1b | 6.22 | 6.35 |
| C2a | 6.65 | 6.73 |
| C2b | 6.67 | 6.74 |
| B1 | 6.72 | 6.78 |
| B2 | 6.73 | 6.78 |

SDS-PAGE

β-mercaptoethanol (or 0.1 M of DTT) was added to SDS-PAGE sample buffer (2× Laemmli sample buffer, Bio-Rad). 50 μL of each diluted sample was mixed with 50 μL of the above SDS-PAGE sample buffer. The tubes were heated at 90° C. for 10 minutes and cooled down to room temperature. The solutions were mixed by vortex mixing. 5 μL of marker (Precision Plus Protein Standard, Unstained, Bio-Rad) was loaded in lane #1 and lane #10. 20 μL of the above solution was loaded in the stain free gels in the lane #2-9 (Mini-Protean TGX stain free precast gels, Any kD, Bio-Rad). The gels were immersed in the TGS running buffer (25 mM Tris-192 mM Glycine-0.1% w/v SDS, pH 8.3). Electrophoresis was performed at 300 V for 18 minutes. Imaging of the gel was done with Gel Doc EZ Imager on a stain free tray (Image Lab 5.1, Bio-Rad).

Fluorescence Measurement

For each diluted time point sample (50 μl sample+950 μl buffer), two 10× and 100× dilutions were prepared with MQ-water. 200 μL from each dilution was loaded in a 96 well plate (black bottom, Thermo scientific). The fluorescence measurement was carried out using an excitation filter of wavelength of 320 nm and recording the emission spectra with a filter of 410 nm in a fluorescence reader (Fluostar Omega, BMG Labtech).

Absorbance Measurement in UV-Vis Spectrophotometer

About 1 mL of each diluted time point sample was carefully transferred to a disposable UV-cuvette and gently tapped to remove any air bubbles. The absorbance at 280 nm and 318 nm was measured using a UV-vis spectrophotometer (UV-1800, Shimadzu). If the absorbance at 280 nm was too high (>2,5), the samples were diluted using MQ-water. The absorbance measurements were also performed in a 96 well plate (MTP) using the Perkin Elmer EnSpire 2300 96 wells plate reader. 100 μl from each time point sample were added into a UV MTP plate. The absorbance at 280 nm and 318 nm was measured using the EnSpire 2300 program.

Activity of HRP or LPO

The activity of HRP or LPO was measured using the 2,2-azino-bis-3-ethylbenzthiazoline-6-sulfonic acid (ABTS) assay at given pH and 40° C. The substrate dosage required for 1 μg/mL of HRP is 10 mM ABTS at given pH and the reaction was started using 0.15% (w/v) H₂O₂. In a first step, 10 μL HRP or LPO or milk was added to the 180 μL ABTS solution and incubated at 40° C. for 10 minutes. Next, 10 μL of H₂O₂ were added and the absorbance at 405 nm measured for 10 minutes (at 40° C.). Enzymatic activity was calculated using the initial slope of ΔA₄₀₅/minute (linear region).

Model Acid Whey and Acid Whey with Readjusted pH

20 mL of model milk was prepared as described in the method above, with and without added Ca²⁺ ions. Small amounts (100 μL/10 mL of milk) of concentrated HCl (12 M) were added to reduce the pH of the milk to 4.6. The precipitate was centrifuged at 5000×g for 15 minutes (20° C.) and the supernatant collected in a separate tube. The pH of the supernatant (model whey) was measured and the supernatant divided into two separate tubes. Concentrated NaOH was used to readjust the pH of one of the tubes to 6.5. In the other tube, a same volume of MQ-water was added. The pH after dilution was measured. The above steps result in 4 different model whey samples (model whey+Ca²⁺, pH˜4.6; model whey+Ca²⁺, pH˜6.5; model whey−Ca²⁺, pH˜4.6; model whey−Ca²⁺, pH˜6.5).

Firmness of Enzymatically Treated Model Yoghurt

Firmness of the model yoghurt was determined using the texture analyzer (XT plus, TA) following the standard protocol known in literature. The skimmed milk (0.1% fat, Arla) was spiked with a fixed conc of LOX (0.15 U/mL), Vanillin (0.5 mM) and 5% of culture (Yoghurt natural Kløvermælk). Different concentrations (0-30 U/mL) of HRP was tested. The control sample contained only the culture. The milk (80 mL) mixture was filled in plastic cups followed by addition and careful mixing of HRP. Next, all the samples were incubated at 43° C. The gelation was visually judged after 6 hours and the samples were stored at 4° C. until analysis by texture analyzer the following day. Before texture analysis, all the samples were placed at 13° C. for approx. 2 hours to adjust the temperature to the measurement value. After texture analysis, the gels were poured out for visual observation.

In another experiment, the milk was first heat treated at 72.5° C. for 40 minutes and then cooled down over ice and stored at 4° C. Next, this heat-treated milk was used for making yoghurt as described above using either single components or a mixture of LOX (0.15 U/mL), Vanillin (0.5 mM) and 5 U/mL of HRP. The final pH reached after 6 hours of fermentation was measured in all samples. The gel firmness was measured as described above.

Phenolic Compounds as Oxidation Mediators

Various low molar mass phenolic compounds were tested as oxidation mediators. The mediators tested were ABTS, Vanillin, Ferulic acid, and p-Coumaric acid. They were tested in skimmed milk (0.1% fat, Arla) in 12 different concentrations and with fixed concentrations of CaCl₂) and LOX, but a high and low concentration of HRP (table 4). The assay was performed in microtiter plates (MTP) incubated at 43° C. for up to 8 hours. The gelation time was inferred from sharp increase in optical density measured at 800 nm. The different milk compositions (1 ml milk with CaCl₂, LOX, HRP and mediators) were prepared in a 2 ml deep well plate, where first the enzymes were added, followed by mediators. All the ingredients were mixed by pipetting and then 100 μl was transferred to MTP plate for reading. The plate was read using the BMG program at an optical density of 800 nm, at 43° C. and the data was collected at an interval of 5 min for up to 8 hours.

TABLE 4
Details of cross-linking reaction conditions for skimmed
milk in the presence of various mediators.

| Parameters | Conc. | Units |
|---|---|---|
| Mediators | 0-1500 | μM |
| HRP | 5 and 30 | U/mL |
| LOX | 0.15 | U/mL |
| CaCl₂ | 20 | g/100 L |

### Example 3: Results

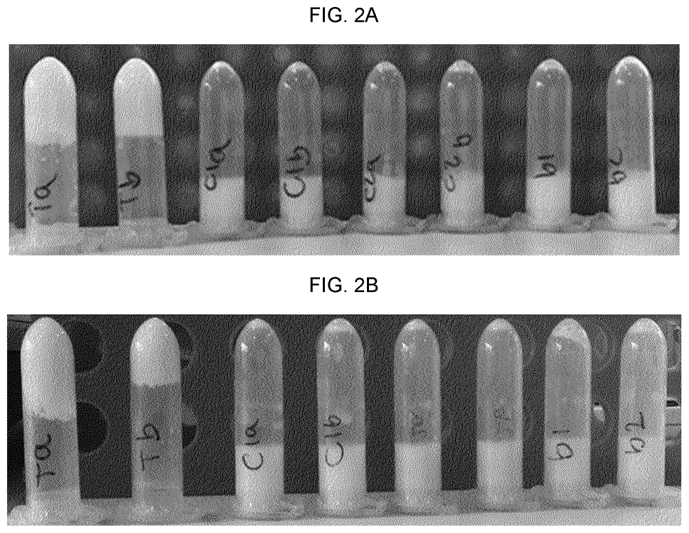

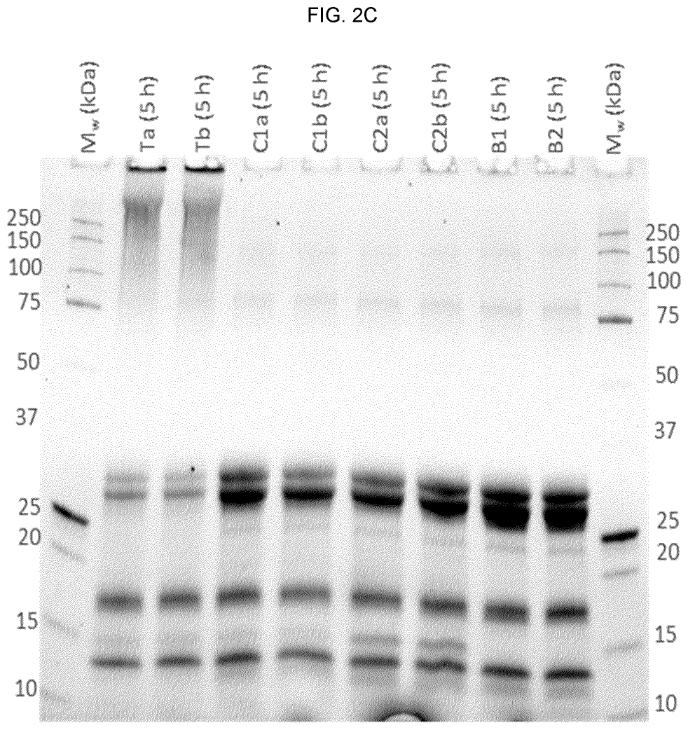

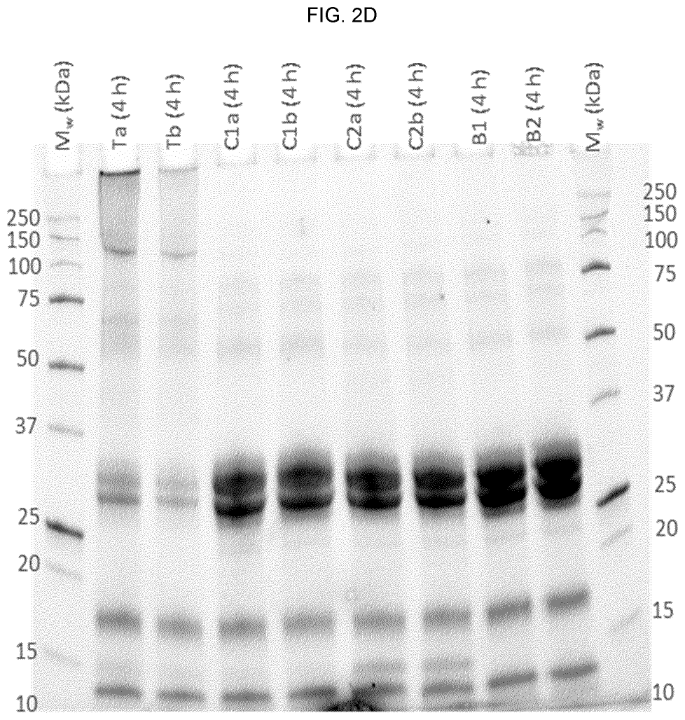

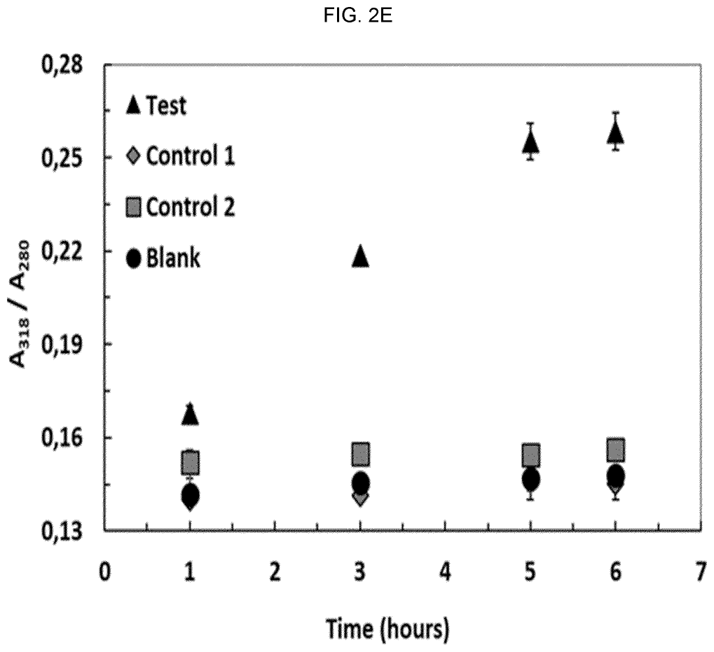

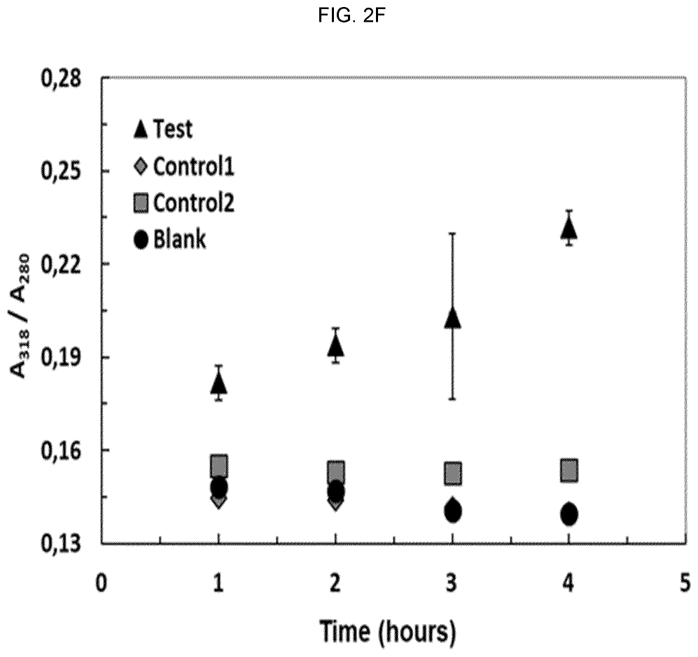

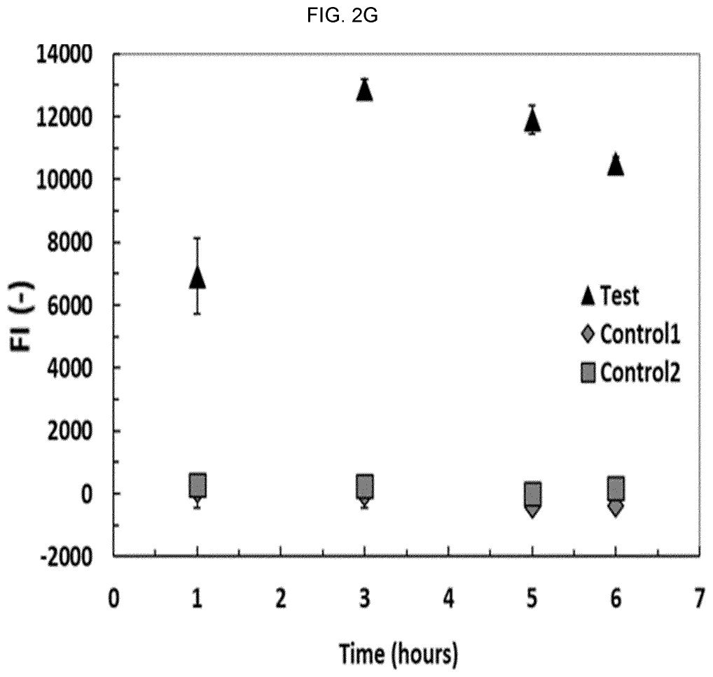

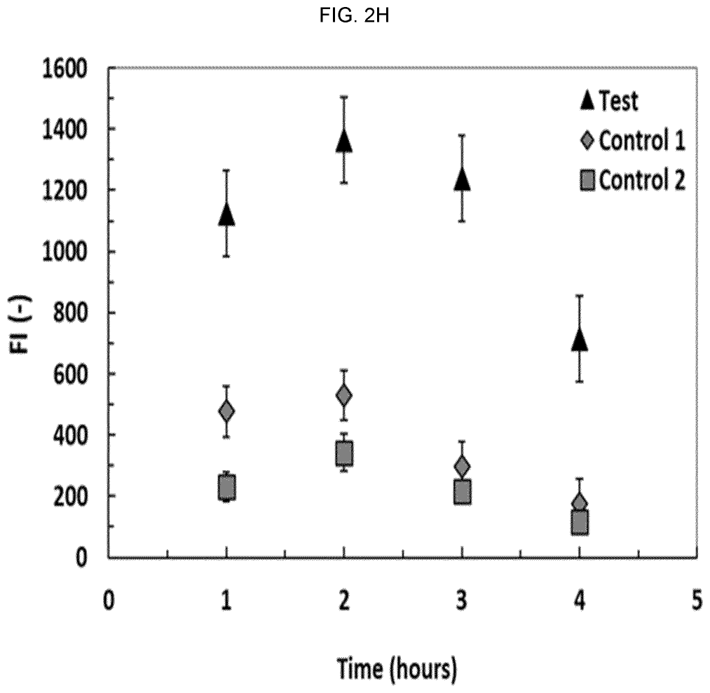

Gelation was observed in the model milk prepared from skimmed milk powder (SMP) after 6 h of incubation with HRP and LOX at 40° C. (FIG. 2 a & b). No gel was formed in the case of blank (without any added enzyme) milk or control milk with either LOX or HRP added. The control with only LOX indicates that gelation was not caused by lowering of pH due to formation of lactobionic acid. The gelation in the test milk (with both LOX and HRP added) is caused by cross-linking of the milk proteins. This was further ascertained from the SDS-PAGE, where high molar mass (>150 kDa) covalently cross-linked polymers were seen for the test samples (FIG. 2 c & d). The monomeric bands (18-30 kDa) progressively decreased with sequential addition of lactose and at the same time, oligomeric bands (30-150 kDa) and polymeric bands (>150 kDa) appeared. The polymer fraction was so large that it did not enter the gel and remained in the pockets. The polymers survived the reducing (DTT), dissociating (SDS) and heating (90° C.) conditions. This observation strongly indicates that the polymerization results from the inter-molecular covalent cross-links other than di-sulfide type of cross-links.

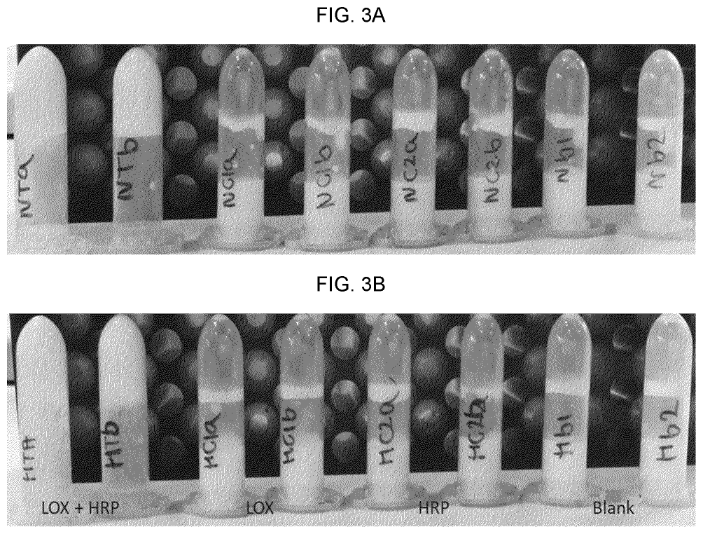

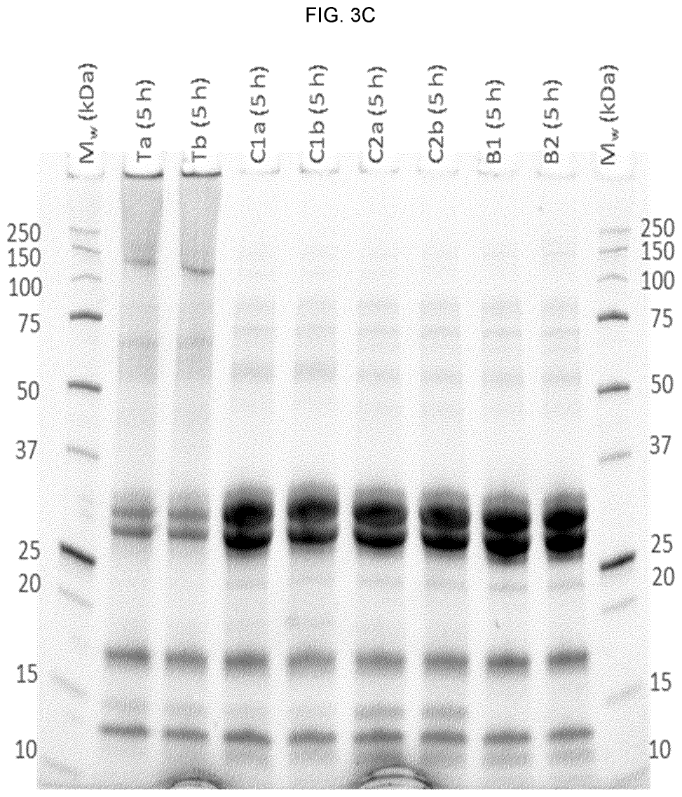

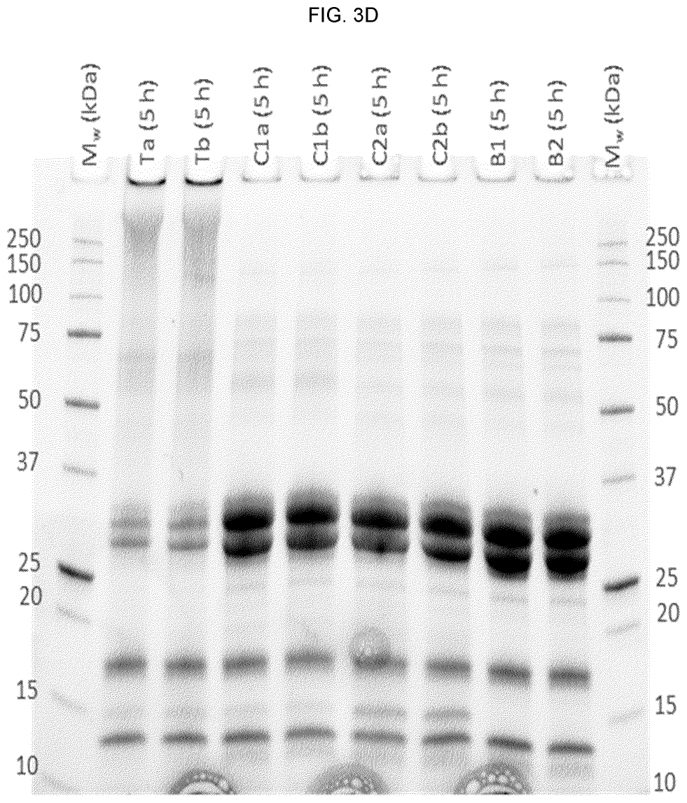

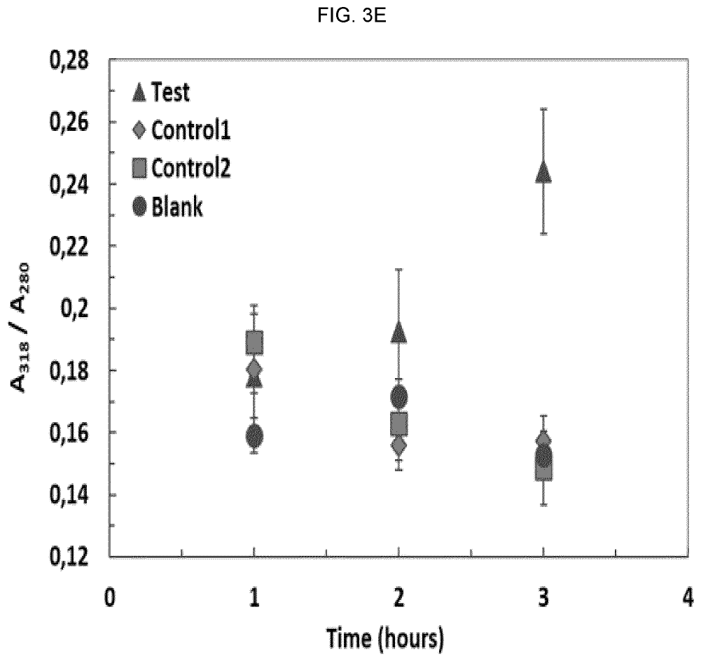

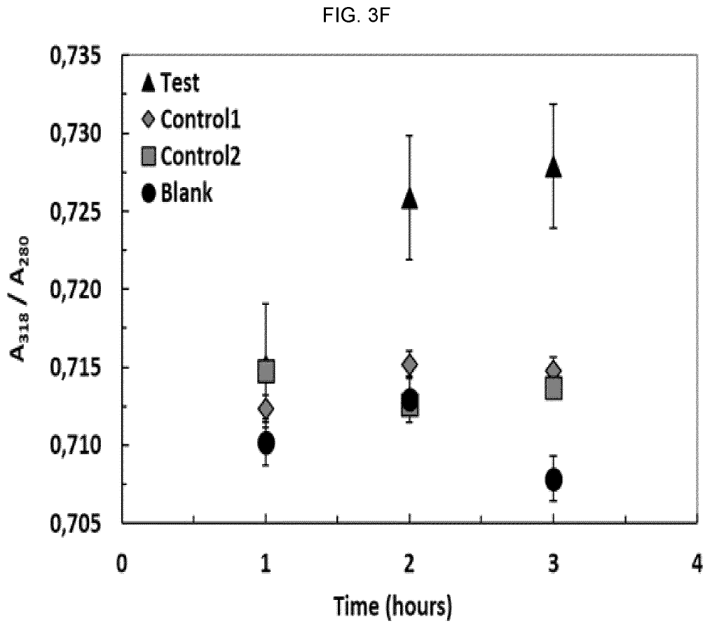

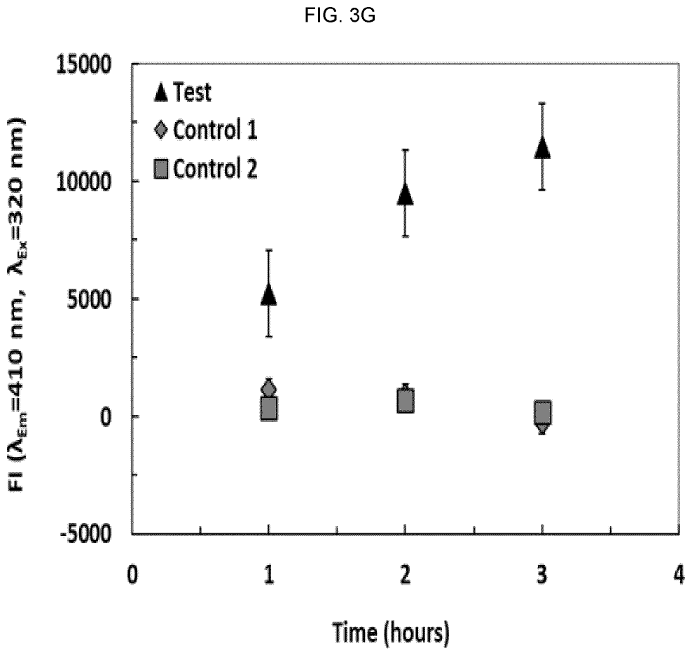

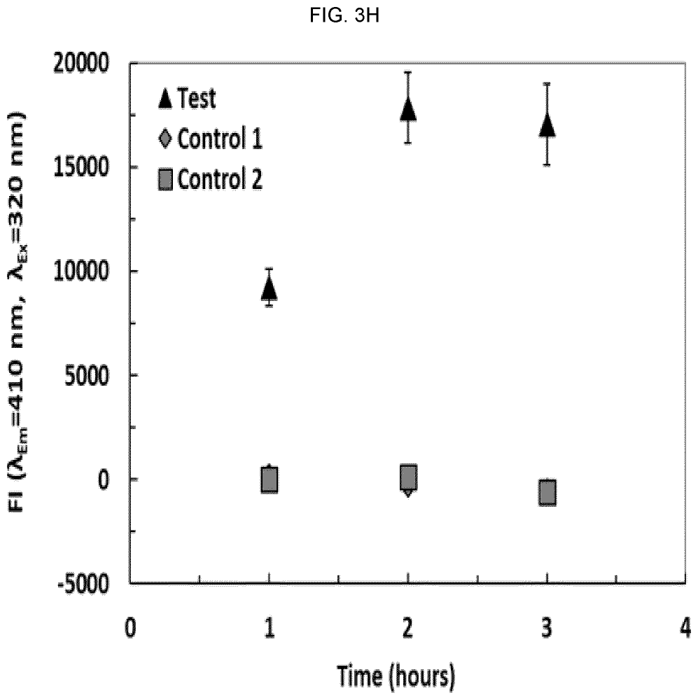

Absorbance and fluorescence measurements were performed to identify the type of cross-links being formed during the polymerization of caseins (FIGS. 2 e-h and 3 e-h). The absorbance at 318 nm (normalized with absorbance at 280 nm) increased in the test samples as compared to the control and blank samples, which indicates formation of oligo-tyrosine (e.g. di-tyrosine) cross-links for the test samples (FIG. 2 e & f). This conclusion was ascertained with the fluorescence measurements. The fluorescence emission spectra after excitation at 320 nm had a peak around 410 nm which is known to be due to di-tyrosine type of cross-links being formed. Increase in fluorescence emission intensity at 410 nm, after excitation at 320 nm for the test samples (FIG. 2 g & h) confirmed the formation of oligo-tyrosine (e.g. di-tyrosine) cross-links. Similar results were obtained in the case of homogenized and non-homogenized real milk with 3.5% fat (FIG. 3 a-h).

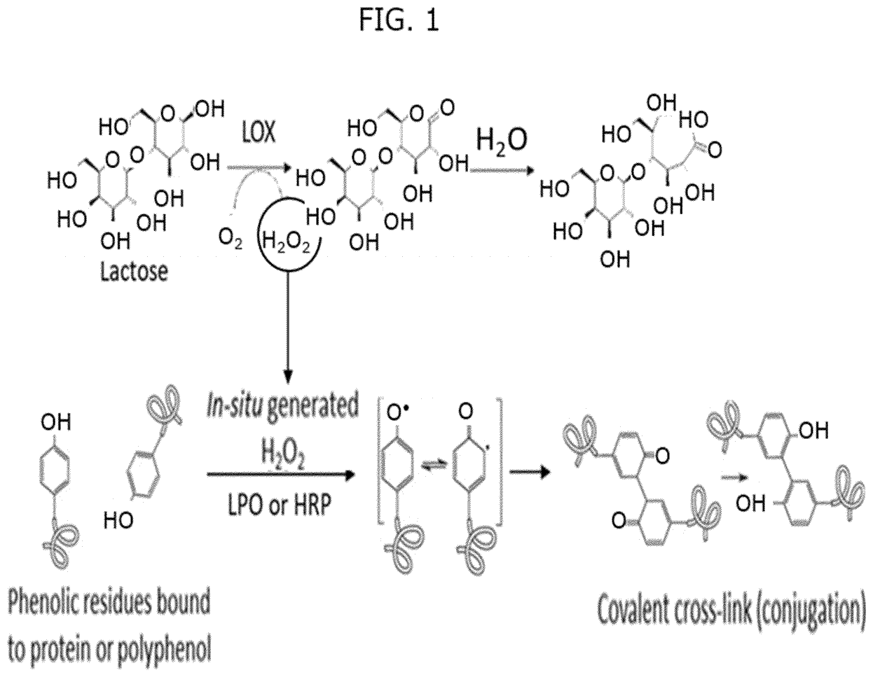

The increase in fluorescence intensity with increasing incubation time was found for the test samples only. This relative fluorescence increase is due to the formation of di-tyrosine (oligo-tyrosine) type of cross-links. The polymerization seems to be of step-growth type, where monomers are converted into dimers, followed by conversion of dimers/monomers into oligomers; eventually cross-linking of oligomers leads to formation of polymers. This can be inferred from the increase and then decrease of the oligomeric fractions in the SDS-PAGE. The fluorescence intensity increase in the homogenized milk was higher than the non-homogenized milk. The di-tyrosine or oligo-tyrosine cross-links being formed can be expected to be present in many different isomeric forms, see FIG. 1 for some isomeric forms of di- and tri-tyrosine. The size (molar mass) of the products being formed by cross-linking of caseins by LOX and HRP can be controlled by controlling the availability of the substrate or by inactivating the enzymes by heat treatment or pH change.

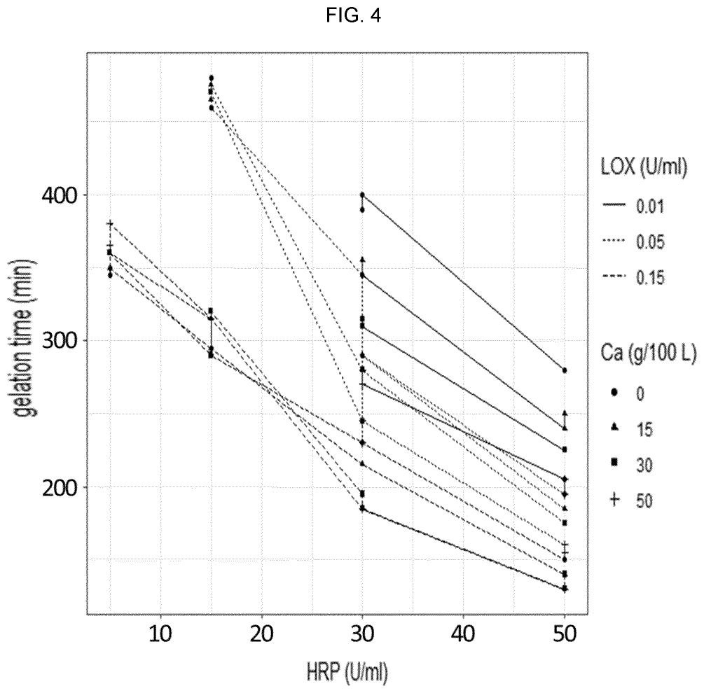

The dosage of Ca²⁺, LOX, and HRP can be varied to control the gelation time of milk (FIG. 4). Samples with HRP dosed at 5 or 15 U/mL did not form a gel within 8 hours if LOX was added at <30 U/mL, irrespective of calcium ion concentration. For samples forming a gel, there was an inverse relationship between peroxidase activity and gelation time (FIG. 4). At low HRP levels there seemed to be an influence of calcium ion concentration, but not at high HRP dosage. The combination of LOX and HRP dosage can be used for controlling the speed of cross-linking.

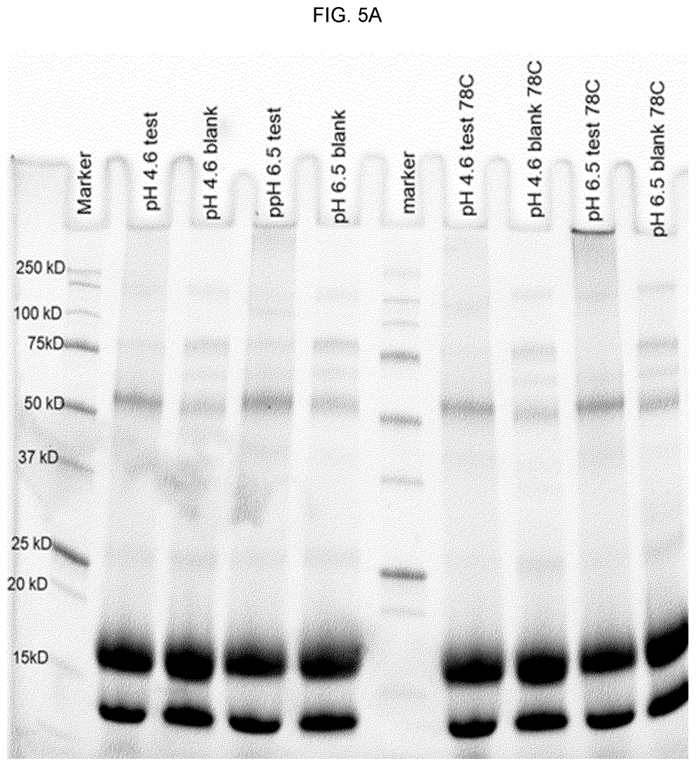

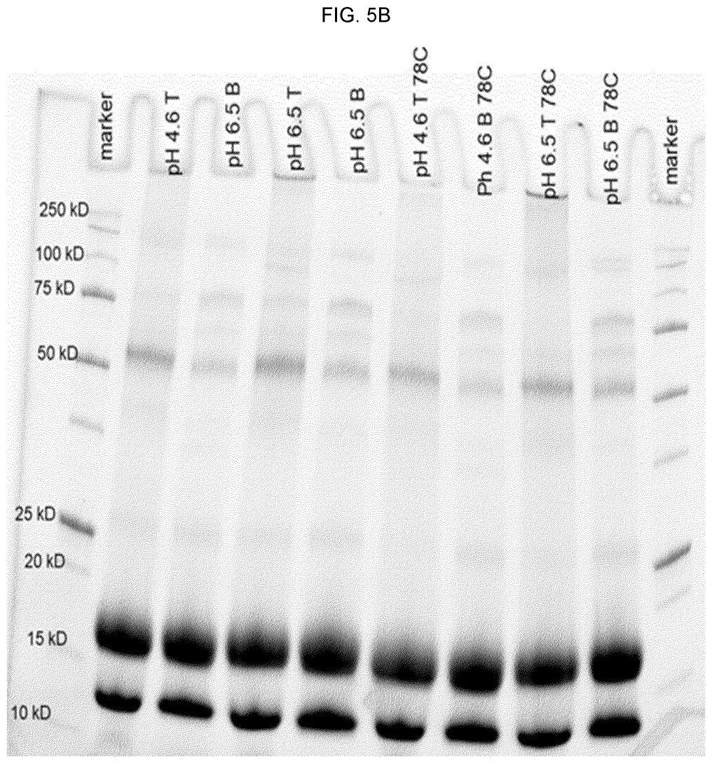

Heat treated whey proteins were found to be cross-linked/polymerized by the combination of LOX and HRP (FIG. 5). The amount of high molar mass (M_{w}>150 kDa) polymers was found to be reduced in the presence of calcium ions. These results indicate that pretreatment, e.g. heating or removal of calcium ions, of globular proteins (such as whey proteins) increases the degree of polymerization. This could be due to a loss of tertiary structure, leading to formation of a molten globule structure which improves the accessibility of the substrate amino acids.

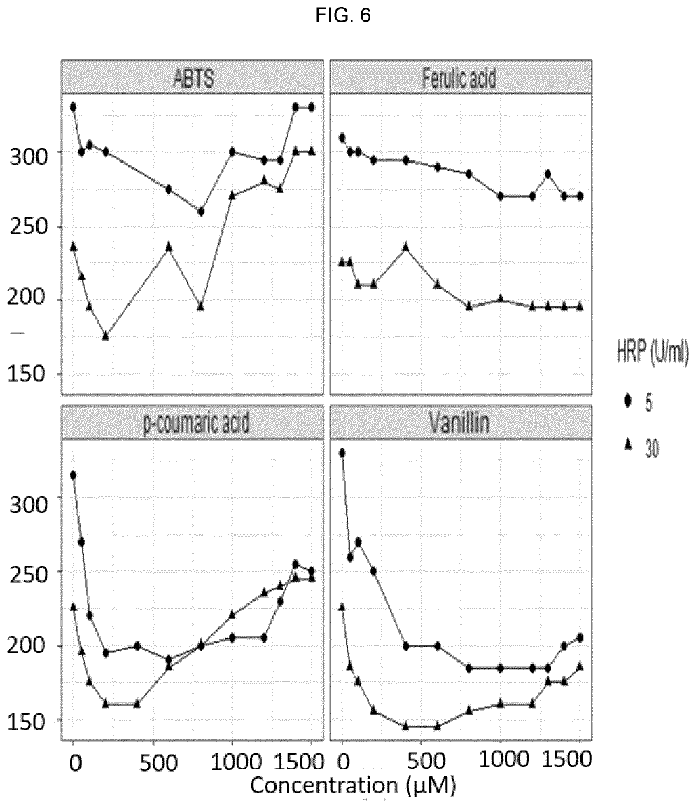

Low molar mass phenolic compounds can act as oxidation mediators in a peroxidase catalyzed reaction. It was found that, both p-coumaric acid and vanillin enhances the oxidation induced cross linking of milk protein (FIG. 6). Using a mediator gives improved control of the enzymatic cross-linking leading to reduction in gelation time. These mediators can be used for switching on/off the cross-linking reaction. For example, dosage of the enzyme can be lowered to a level where it does not influence texture without the mediator. The phenol type mediators are not stable and will over time self-polymerize into an inactive homopolymer. Therefore, by controlling concentrations of enzyme and mediator one can control the life time of the enzyme reaction, and not having to deal with a cross linking reaction that will just continue after optimal texture has been attained. Vanillin is the active compound in synthetic vanilla aroma which is used in many food products.

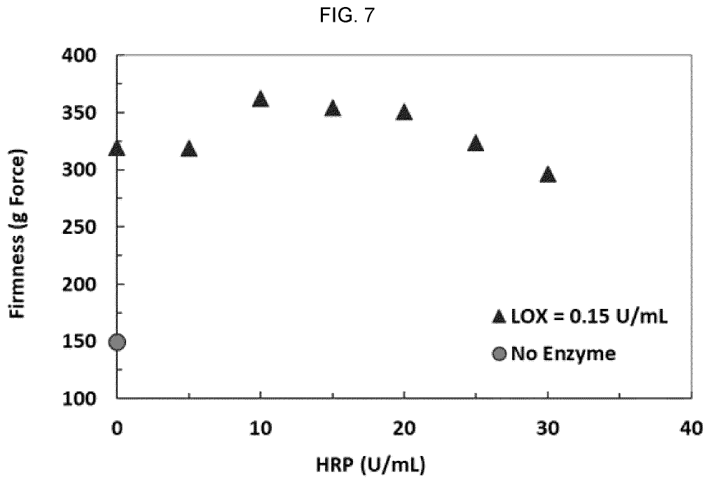

Fermentation of skimmed milk (0.1% fat) with simultaneous addition of LOX (0.15 U/mL) and vanillin (0.5 mM) resulted in a firmer yoghurt as compared to the control i.e. without LOX and vanillin (FIG. 7). The texture of the control was visibly less than that of the enzymatically treated samples, even though the final pH in all the samples had reached around 4.6. The results with the addition of only LOX indicates that some of the lactoperoxidase (LPO) in the milk might have still been active, even after pasteurization. In addition, there could be texturization effect caused by the Ca-lactobionate, formed by complexing of free Ca²⁺ ions in the milk and the lactobionic acid generated by the action of LOX on lactose. Combination of LOX and vanillin with a varying dosage of HRP (5-30 U/mL) resulted in a visibly increased firmness of the yoghurt up to 20 U/mL of HRP dosage. Higher activity dosage of HRP did not seem to give any addition benefit in terms of firmness. This could be due to the graininess created by excessive cross-linking, as typically observed in the case of transglutaminase.

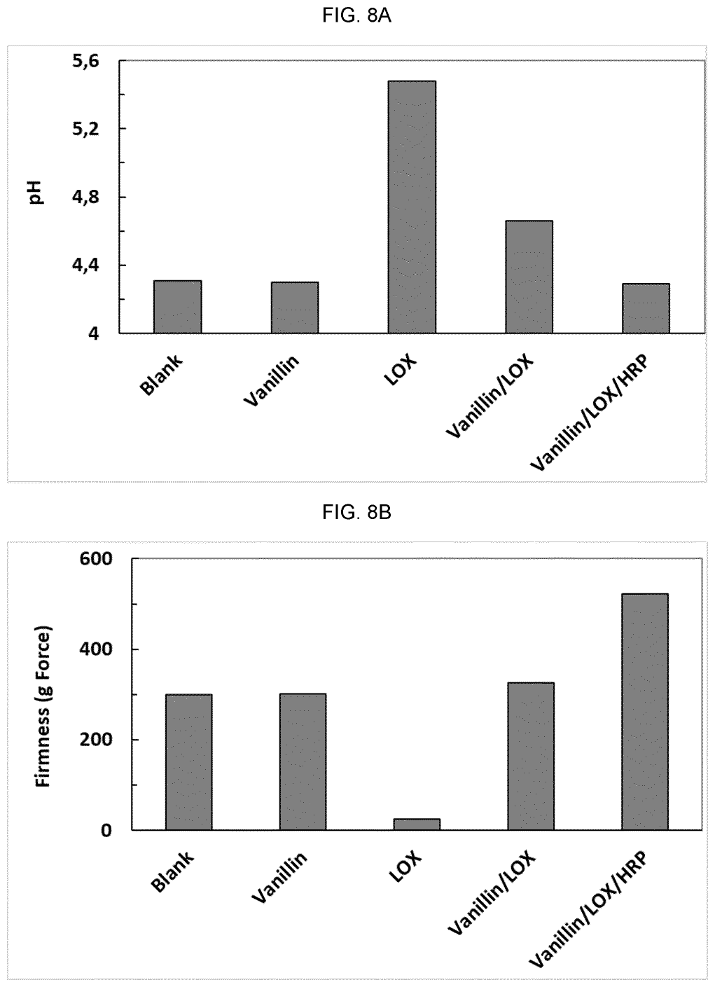

To further probe the effect of adding enzymes on acidification and texturization, the heat-treated milk was used for making the yoghurt. The acidification was slowed down in the case of only LOX added to heat-treated milk (FIG. 8A). The final pH value at the end of incubation was ˜5.4 as compared to ˜4.3 for the blank. This indicates that the H₂O₂ generated after the addition of LOX may in some cases be detrimental to some of the culture cells which in turn leads to slower acidification. Incorporation of an oxidation mediator partly mitigates this negative effect since it acts as a scavenger for the H₂O₂ in presence of the lactoperoxidase (LPO) from milk (FIG. 8A). Another possibility to bypass the negative effect of H₂O₂ produced with the addition of LOX is to do the enzymatic treatment before the culture addition or after the acidification by cultures. However, in the presence of HRP, there was no negative effect observed on acidification of the heat treated milk (FIG. 8A). This indicates that phenolic residues e.g. phenolic mediators or the aromatic amino acids on proteins such as tyrosine are somewhat better substrates for the enzymatic reaction, so there is no free H₂O₂ left that could inactivate the culture cells. In fact, when milk was incubated with both the LOX and HRP, the acidification kinetics were same as that of blank and the texture of the final yoghurt was significantly higher than that of the blank yoghurt (FIG. 8B).

### ITEMS

1. A method for producing a modified food product comprising at least one cross-linked compound, said method comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with at least one oxidase selected from a cellobiose oxidase (EC 1.1.99.18) and a hexose oxidase such as a glucose oxidase (EC 1.1.3.4) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   thereby obtaining a modified food product comprising at least one cross-linked compound.
2. A method for modifying a property such as firmness and/or gelation time of a food product, comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with at least one oxidase selected from a cellobiose oxidase (EC 1.1.99.18) and a hexose oxidase such as a glucose oxidase (EC 1.1.3.4) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   thereby obtaining a modified food product having increased firmness and/or reduced gelation time compared to the firmness and/or gelation time of the substrate.
3. A method for producing a modified food product comprising at least one cross-linked compound, said method comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   thereby obtaining a modified food product comprising at least one cross-linked compound.
4. A method for modifying a property such as firmness and/or gelation time of a food product, comprising the steps of:
i) providing a substrate comprising oxygen and a carbohydrate substrate such as lactose and at least one first compound selected from a phenolic compound, a non-phenolic aromatic compound, a compound comprising a sulfhydryl group and a compound comprising an amino group, such as a protein comprising at least one aromatic amino acid such as tyrosine, wherein the substrate is the food product to be modified;
ii) contacting said substrate with a cellobiose oxidase (EC 1.1.99.18) and with a peroxidase (EC 1.11.1.7);
iii) incubating said substrate with said cellobiose oxidase and with said peroxidase, whereby said cellobiose oxidase catalyzes conversion of the carbohydrate substrate and oxygen into a corresponding organic acid and H₂O₂ in the substrate, and whereby said peroxidase catalyzes cross-linking of said first compound using said H₂O₂ as a co-substrate;
   thereby obtaining a modified food product having increased firmness and/or reduced gelation time compared to the firmness and/or gelation time of the substrate.
5. The method according to any one of the preceding items, wherein the oxidase is a cellobiose oxidase.
6. The method according to any one of the preceding items, wherein the carbohydrate substrate is lactose and the acid is lactobionic acid, or wherein the carbohydrate substrate is glucose and the acid is gluconic acid, or wherein the carbohydrate substrate is galactose and the acid is galactonic acid, or wherein the carbohydrate substrate is maltose and the acid is maltobionic acid, or wherein the carbohydrate substrate is xylose and the acid is xylonic acid, or wherein the carbohydrate substrate is cellobiose and the acid is cellobionic acid, or wherein the carbohydrate substrate is mannose and the acid is mannonic acid, or wherein the carbohydrate substrate is fructose and the acid is fructonic acid, preferably the carbohydrate substrate is lactose and the acid is lactobionic acid.
7. The method according to any one of the preceding items, wherein the carbohydrate substrate is lactose and the acid is lactobionic acid, or wherein the carbohydrate substrate is glucose and the acid is gluconic acid, or wherein the carbohydrate substrate is galactose and the acid is galactonic acid, preferably the carbohydrate substrate is lactose and the acid is lactobionic acid.
8. The method according to any one of the preceding items, wherein the cross-linking comprises the formation of intramolecular and/or intermolecular covalent cross-links between molecules of the first compound.
9. The method according to any one of the preceding items, wherein the first compound is a phenolic compound.
10. The method according to any one of the preceding items, wherein the cross-linking comprises the formation of oligo-tyrosine cross-links, such as the formation of di-tyrosine cross-links and/or iso-dityrosine cross-links and/or disulphide cross-links and/or cross-links formed by covalent bonds of type C—C, C—O—C, C—N, C—S, S—S, wherein covalent cross-links are formed enzymatically and/or non-enzymatically.
11. The method according to any one of the preceding items, wherein the peroxidase is endogenous to the substrate, preferably the peroxidase is lactoperoxidase.
12. The method according to any one of the preceding items, wherein the peroxidase is exogenous to the substrate, preferably wherein the peroxidase is lactoperoxidase.
13. The method according to any one of the preceding items, wherein the peroxidase is lactoperoxidase, horseradish peroxidase, lignin peroxidase, Coprinus peroxidase or myeloperoxidase, preferably lactoperoxidase or horseradish peroxidase, most preferably lactoperoxidase.
14. The method according to any one of the preceding items, wherein the substrate is a dairy product.
15. The method according to any one of the preceding items, wherein the substrate is a yogurt, quark, a cheese such as a soft cheese, a drinking yogurt, a cheese spread, skyr or milk, such as soy milk, sheep milk, goat milk, buffalo milk, yak milk, lama milk, camel milk or cow milk, or a combination thereof, optionally supplemented with plant material.
16. The method according to any one of the preceding items, wherein the first compound is a protein such as a casein or whey protein.
17. The method according to any one of the preceding items, wherein if the first compound is whey protein, the method further comprises a step of pre-treatment of the substrate prior to step iii), wherein the step of pre-treatment is a step of heat treatment, a step of reduction of disulphide bridges and/or a step of removal of multivalent ions, whereby accessibility of the at least one aromatic amino acid is increased.
18. The method according to any one of the preceding items, wherein the substrate is a dairy product comprising lactose, and wherein the method further comprises contacting and incubating the substrate with a lactase prior to step i), or during any of steps i), ii) and iii), preferably prior to or during step i), whereby the lactase converts the lactose to galactose and glucose, and wherein the oxidase in step iii) catalyzes conversion of the galactose into galactonic acid and H₂O₂ and/or conversion of the glucose into gluconic acid and H₂O₂, and wherein the oxidase is preferably a cellobiose oxidase.
19. The method according to any one of the preceding items, wherein the substrate is a dairy product comprising lactose, and wherein the method further comprises incubating the substrate with a lactase prior to step i), wherein the lactase converts the lactose to galactose and glucose, and wherein the oxidase in step iii) catalyzes conversion of the galactose into galactonic acid and H₂O₂ and/or conversion of the glucose into gluconic acid and H₂O₂, and wherein the oxidase is preferably a cellobiose oxidase.
20. The method according to any one of the preceding items, wherein the substrate comprises in the range of 0.01% to 30% w/w of the first compound, such as 0.05%, 1%, 5%, 10%, 15%, 20%, 25% w/w, for example between 2.5 and 6% w/w, such as 3.5% w/w.
21. The method according to any one of the preceding items, wherein the substrate comprises in the range of 0.01% to 30% w/w of carbohydrate substrate, preferably wherein the carbohydrate substrate is lactose, such as 0.05%, 1%, 5%, 10%, 15%, 20%, 25% w/w, for example between 2.5 and 6% w/w, such as 4.5% w/w.
22. The method according to any one of the preceding items, wherein the concentration of oxidase relative to the substrate is in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.03 and 7.5 U/g substrate or between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.
23. The method according to any one of the preceding items, wherein the oxidase is a cellobiose oxidase and the concentration of cellobiose oxidase relative to the substrate is in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.03 and 7.5 U/g substrate or between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.
24. The method according to any one of the preceding items, wherein the oxidase is a hexose oxidase such as a glucose oxidase and the concentration of hexose oxidase, such as the concentration of glucose oxidase, relative to the substrate is in the range of 0.0001 to 15 U/g substrate, such as 0.01 U/g substrate, 0.05 U/g substrate, or 0.15 U/g substrate, for example between 0.001 and 12.5 U/g substrate, such as between 0.005 and 10 U/g substrate, for example between 0.01 and 7.5 U/g substrate, such as between 0.03 and 7.5 U/g substrate or between 0.05 and 5 U/g substrate, for example between 0.1 and 2.5 U/g substrate, such as between 0.15 and 1 U/g substrate, for example between 0.25 and 0.75 U/g substrate, such as 0.5 U/g substrate.
25. The method according to any one of the preceding items, wherein the concentration of peroxidase relative to the substrate is in the range of 0.001 to 500 U/g substrate, such as 5, 15, 30, or 50 U/g substrate, for example between 0.01 and 250 U/g substrate, such as between 0.05 and 125 U/g substrate, for example between 0.1 and 100 U/g substrate, such as between 0.5 and 75 U/g substrate, for example between 1 and 50 U/g substrate, such as between 5 and 40 U/g substrate, for example between 10 and 30 U/g substrate, for example 15, 20 or 25 U/g substrate.
26. The method according to any one of the preceding items, wherein step iii) is performed at a temperature of 4° C. to 75° C., such as between 4° C. and 72° C., for example between 4° C. and 70° C., such as between 4° C. and 65° C., for example between 4° C. and 60° C., such as between 4° C. and 55° C., for example between 4° C. and 50° C., such as between 4° C. and 45° C., for example between 4° C. and 40° C., such as between 4° C. and 37° C., for example between 4° C. and 35° C., such as between 4° C. and 30° C., for example between 4° C. and 25° C., such as between 4° C. and 20° C., for example between 4° C. and 15° C., such as between 4° C. and 10° C., or such as between 10° C. and 75° C., for example between 15° C. and 75° C., such as between 20° C. and 75° C., for example between 25° C. and 75° C., such as between 30° C. and 75° C., for example between 35° C. and 75° C., such as between 37° C. and 75° C., for example between 40° C. and 75° C., such as between 45° C. and 75° C., for example between 50° C. and 75° C., such as between 55° C. and 75° C., for example between 60° C. and 75° C., such as between 65° C. and 75° C., for example between 72° C. and 75° C., such as at 75° C., 72° C., 40° C., 37° C., 25° C. or 4° C.
27. The method according to any one of the preceding items, wherein step iii) is performed for a duration of between 15 seconds and 144 hours, such as between 30 seconds and 132 hours, for example between 1 minute and 120 hours, such as between 2 minutes and 108 hours, for example between 5 minutes and 96 hours, such as between 10 minutes and 84 hours, for example between 20 minutes and 72 hours, such as between 30 minutes and 60 hours, for example between 1 hour and 48 hours, such as between 2 hours and 44 hours, for example between 3 hours and 40 hours, such as between 3 hours and 36 hours, for example between 4 hours and 32 hours, such as between 4 hours and 28 hours, for example between 5 hours and 24 hours, such as between 5 hours and 20 hours, for example between 6 hours and 16 hours, such as between 6 hours and 12 hours, for example between 1 hour and 10 hours, such as between 2 hours and 8 hours, for example between 3 hours and 6 hours, such as 3, 4, 5 or 6 hours.
28. The method according to any one of the preceding items, wherein step iii) is performed at a temperature of 75° C. for 15 seconds, or at a temperature of 72° C. for 30 seconds, or at a temperature of 40° C. for 3 to 6 hours, such as at a temperature of 40° C. for 3 hours, for 4 hours, for 5 hours or for 6 hours.
29. The method according to any one of the preceding items, wherein the pH of the substrate in any of steps i), ii) or iii) and/or the pH of the product in step iii) is in the range of 3.5 to 8.5, such as between 4.0 and 8.0, for example between 4.5 and 7.5, such as between 5.0 and 7.2, for example between 5.5 and 7.0, such as between 6.0 and 6.9, for example between 6.2 and 6.8, such as between 6.4 and 6.7, for example 6.6.
30. The method according to any one of the preceding items, further comprising providing an additional substrate comprising at least one co-mediator which consists of Ca²⁺ or a second compound such as a phenolic compound, and contacting and incubating said additional substrate with the substrate in steps ii) and iii), whereby the cross-linking in step iii) comprises the formation of intermolecular covalent cross-links between molecules of the first compound of the substrate, and/or between molecules of the second compound, and/or between molecules of the first compound comprised in the substrate and molecules of the second compound.
31. The method according to any one of the preceding items, wherein the additional substrate is a grain hull, a grain such as a cereal grain, fruit pulp or fruit peel, a bean such as a coffee bean, a leaf such as a tea leaf, a vegetable pulp or a vegetable peel such as pulp or peel from a tuberculous vegetable, a fruit extract, a vegetable extract, a seed extract or a yeast extract.
32. The method according to any one of the preceding items, wherein the co-mediator is selected from the group consisting of caffeic acid, cholorogenic acid, flavonoids, flavonols, quercetin, rutin, tannic acid, vanillin, p-coumaric acid and ferulic acid.
33. The method according to any one of the preceding items, wherein the co-mediator is Ca²⁺, preferably wherein the concentration of Ca²⁺ is between 0.05 and 5000 mg/L, such as between 0.1 and 4000 mg/L, for example between 10 and 3000 mg/L, such as 100 and 2500 mg/L, for example between 150 and 2000 mg/L, such as between 300 and 1500 mg/L, for example between 500 and 1000 mg/L, such as between 600 and 900 mg/L, for example between 700 and 800 mg/L.
34. The method according to any one of the preceding items, further comprising a step of heating the modified food product to inactivate the oxidase and/or the peroxidase, such as heating at 90° C. for 10 minutes or heating at 141° C. for 8 seconds or heating at 72° C. for 15 seconds or heating at 63° C. for 30 minutes.
35. The method according to any one of the preceding items, further comprising a step of reducing the pH of the modified food product to below 4, whereby the oxidase and/or the peroxidase is inactivated.
36. The method according to any one of the preceding items, wherein steps ii) and/or iii) are performed concomitantly with a step of fermentation, such as fermentation of milk to a dairy product, and/or with a step of bacterial acidification.
37. The method according to any one of the preceding items, further comprising a step of pasteurization or sterilization.
38. The method according to any one of the preceding items, wherein the modified food product comprises at least 0.001% cross-linked compound, such as at least 0.01%, such as at least 0.1%, such as at least 0.5%, such as at least 1%, such as at least 2% cross-linked compound, such as at least 5%, such as at least 10%, such as at least 20%, such as at least 30%, such as at least 40%, such as at least 50%, such as at least 60%, such as at least 70% or more, wherein the percentage is in w/w of total protein of the food product.
39. The method according to any one of the preceding items, wherein the food product comprises 0.00001 mg to 250 mg of cross-linked compound per g of food product, such as from 0.0001 to 200 mg, such as from 0.001 to 150 mg, such as from 0.01 to 100 mg, such as from 0.1 to 75 mg, such as from 0.5 to 74 mg, such as from 1 to 50, such as from 5 to 25 mg of cross-linked compound per g of food product.
40. The method according to any one of the preceding items, wherein the averaged degree of polymerisation (DP) of the cross-linked compound is from 2 to 100000, such as from 3 to 100000, such as from 5 to 1000, such as from 8 to 200, such as from 9 to 150, such as 100 or 125.
41. A modified food product obtainable by the method according to any one of the preceding items.
42. The modified food product according to item 41, wherein at least one property of the modified food product is modified compared to the corresponding properties of the substrate.
43. The modified food product according to any one of items 41 to 42, wherein the modified food product has a shorter gelation time, an increased firmness, or reduced likelihood of syneresis compared to the substrate.
44. The modified food product according to any one of items 41 to 43, wherein the modified food product is a dairy product such as a yogurt, quark, a cheese such as a soft cheese, a drinking yogurt, a cheese spread, skyr or milk, such as soy milk, sheep milk, goat milk, buffalo milk, yak milk, lama milk, camel milk or cow milk, or a combination thereof, optionally supplemented with plant material.

1-15. (canceled)

16. A method for one or more of improving firmness of a food product, improving gelation time of a food product, and reducing likelihood of syneresis in a food product, comprising:
(a) contacting (i) a substrate food product comprising oxygen, a carbohydrate, and a first compound selected from a phenolic compound, a non-phenolic aromatic compound, a compound comprising a sulfhydryl group, and a compound comprising an amino group with (ii) an oxidase selected from a cellobiose oxidase (EC 1.1.99.18) and a hexose oxidase and with (iii) a peroxidase (EC 1.11.1.7), and
(b) incubating said substrate with said oxidase and said peroxidase, whereby said oxidase catalyzes conversion of said carbohydrate and oxygen into a corresponding organic acid and H₂O₂ and said peroxidase catalyzes cross-linking of said first compound with said H₂O₂ acting as a co-substrate;
thereby obtaining a modified food product having one or more of increased firmness, reduced gelation time, and reduced likelihood of syneresis relative to the substrate food product.

17. The method according to claim 16, wherein one or more of:
the carbohydrate is lactose and the corresponding organic acid is lactobionic acid;
the carbohydrate is glucose and the corresponding organic acid is gluconic acid;
the carbohydrate is galactose and the corresponding organic acid is galactonic acid;
the carbohydrate is maltose and the corresponding organic acid is maltobionic acid;
the carbohydrate is xylose and the corresponding organic acid is xylonic acid;
the carbohydrate is cellobiose and the corresponding organic acid is cellobionic acid;
the carbohydrate is mannose and the corresponding organic acid is mannonic acid; and
the carbohydrate is fructose and the corresponding organic acid is fructonic acid.

18. The method according to claim 16, wherein the carbohydrate is lactose and the corresponding organic acid is lactobionic acid.

19. The method according to claim 16, wherein the oxidase is a glucose oxidase (EC 1.1.3.4).

20. The method according to claim 16, wherein the oxidase is a cellobiose oxidase (EC 1.1.99.18).

21. The method according to claim 16, wherein the first compound is a phenolic compound.

22. The method according to claim 16, wherein the first compound is a protein comprising a tyrosine residue.

23. The method according to claim 16, wherein the peroxidase is selected from lactoperoxidase, horseradish peroxidase, lignin peroxidase, Coprinus peroxidase, and myeloperoxidase.

24. The method according to claim 16, wherein the peroxidase is lactoperoxidase.

25. The method according to claim 16, wherein the substrate food product is selected from yogurt, quark, cheese, drinking yogurt, a cheese spread, skyr, a milk, and soy milk, optionally supplemented with plant material.

26. The method according to claim 16, wherein substrate food product is a dairy product comprising lactose, and wherein the method further comprises incubating the substrate food product with a lactase prior to or during step (a), whereby the lactase converts the lactose to galactose and glucose, and the oxidase catalyzes one or both of (1) conversion of the galactose into galactonic acid and H₂O₂ and (2) conversion of the glucose into gluconic acid and H₂O₂.

27. The method according to claim 16, wherein the concentration of the oxidase relative to the substrate food product is from 0.0001 to 15 U/g substrate.

28. The method according to claim 16, wherein the concentration of peroxidase relative to the substrate food product is from 0.001 to 500 U/g substrate.

29. The method according to claim 16, further comprising contacting and incubating the substrate food product with an additional substrate comprising a co-mediator selected from Ca⁺ and a second compound, whereby the peroxidase catalyzes one or more of (1) formation of intermolecular covalent cross-links between molecules of the first compound, (2) formation of intermolecular covalent cross-links between molecules of the second compound, and (3) formation of intermolecular covalent cross-links between molecules of the first compound and molecules of the second compound, wherein the second compound is a phenolic compound.

30. The method of claim 29, wherein the co-mediator is selected from caffeic acid, cholorogenic acid, flavonoids, flavonols, quercetin, rutin, tannic acid, vanillin, p-coumaric acid, and ferulic acid.

31. The method of claim 29, wherein the co-mediator is selected from vanillin and p-coumaric acid.

32. The method according to claim 29, wherein the co-mediator is Ca⁺.

33. The method according to claim 32, wherein the Ca⁺ is provided at concentration of from 0.05 to 5000 mg/L.

34. The method according to claim 16, further comprising one or more of:
inactivating one or both of the oxidase and the peroxidase by heating the modified food product;
inactivating one or both of the oxidase and the peroxidase by reducing the pH of the modified food product to below 4;
fermentation of the substrate food product, optionally simultaneously with step (b);
bacterial acidification of the substrate food product, optionally simultaneously with step (b);
pasteurization of the modified food product; and
sterilization of the modified food product.

35. The method according to claim 16, wherein the modified food product comprises at least 0.001% w/w cross-linked compound, based on the total protein content of the modified food product.

36. A modified food product obtained by the method of claim 16, wherein the modified food product has one or more of shorter gelation time, increased firmness, or reduced likelihood of syneresis relative to the substrate food product.
